(19) United States
(12) Patent Application Publication
(10) Pub. No.: US 2023/0365930 A1
(43) Pub. Date: Nov. 16, 2023

(54) IMMUNOLOGICAL ENHANCEMENT OF STEM CELL ACTIVITY IN TREATMENT OF OVARIAN FAILURE

(71) Applicant: CREATIVE MEDICAL TECHNOLOGIES, INC., Phoenix, AZ (US)

(72) Inventors: Thomas ICHIM, San Diego, CA (US); Amit PATEL, Salt Lake City, UT (US); Courtney BARTLETT, Niceville, FL (US)

(73) Assignee: CREATIVE MEDICAL TECHNOLOGIES, INC., Phoenix, AZ (US)

(21) Appl. No.: 18/311,457

(22) Filed: May 3, 2023

Related U.S. Application Data
(60) Provisional application No. 63/340,454, filed on May 10, 2022.

(51) Int. Cl. C12N 5/0783

(52) U.S. Cl. CPC C12N 5/0636; C12N 2501/71

(57) ABSTRACT

Disclosed are methods, protocols and compositions of matter using for treatment of ovarian failure through administration of cells expressing FoxP3 as a monotherapy or as a facilitator to enhance therapeutic efficacy of cells and/or cytokines. In one embodiment FoxP3 expressing cells are concentrated from allogeneic umbilical cord blood, activated ex vivo with anti-CD3 and anti-CD28 antibodies and administered into the ovary alone or with regenerative cells such as stem cells. In one embodiment stem cells administered are of the CD105 expressing lineage.

### CROSS-REFERENCE TO RELATED APPLICATIONS

This application claims priority to U.S. Provisional Application No. 63/340,454, titled “Immunological Enhancement of Stem Cell Activity in Treatment of Ovarian Failure” filed May 10, 2022, which is hereby incorporated by reference herein in its entirety.

### FIELD OF THE INVENTION

The invention is directed to methods of treating and preventing ovarian failure by administering regenerative cells to a patient in need.

### BACKGROUND

Currently there is no treatment for ovarian failure. Various experimental treatments have been developed, with early preliminary data existing, however, to date, none of passed formal clinical trials in a double-blind manner.

Zhang et al conducted a study in which female mice were subjected to superovulation and ozone inhalation to create a model of accelerated ovarian aging with a decline in both the quantity and quality of oocytes. Cells were transplanted via IV or MI, and ovaries were removed after 2 weeks or 1 month of treatment. Ovarian reserve and function were evaluated based on the follicle counts, hormone levels, the estrous cycle, and reproductive performance. Cell tracking, terminal deoxynucleotidyl transferase-mediated dUTP nick end labeling (TUNEL), real-time polymerase chain reaction (PCR), and Western blot analysis were used to assess the inner mechanisms of injury and repair. The results indicated that ovarian function increased significantly after treatment with hUCMSCs. Immunofluorescence revealed reduced TUNEL staining and a decreased percentage of apoptotic cells. A higher level of expression of anti-apoptotic and antioxidant enzymes was noted in the ovaries of groups treated with hUCMSCs. These parameters were enhanced more when mice were treated with hUCMSCs for 1 month than when they treated with hUCMSCs for 2 weeks. IV was better able to restore ovarian function than MI. These results suggest that both methods of transplantation may improve ovarian function and that IV transplantation of hUCMSCs can significantly improve ovarian function and structural parameters more than MI transplantation of hUCMSCs can [1]. In a chemotherapy associated ovarian failure model, one study examined a total of 110 female BALB/c mice (aged 7-8 wk old) with body masses of 16.0-20.0 g. The mice were fed until 12 wk of age, and the chemotherapy cyclophosphamide was administered by intraperitoneal injection for 14 consecutive days to induce premature ovarian failure in mice. Seventy-five mice with estrous cycle disorder were screened and randomly divided into 3 groups according to their body weight:model group, positive control group and hUCMSC group, and each group had 25 mice. Another 25 mice were used as negative controls. The mice in the hUCMSC group were injected with hUCMSCs in the tail vein, and the mice in the positive control group were given an oestradiol valerate solution and a medroxyprogesterone acetate solution in the tail vein. On the 1^{st}, 15^{th}, 30^{th}, 45^{th}, and 60^{th} days after intravenous administration, vaginal smears were made to monitor the estrous cycles of the mice. The ovaries were weighed, and pathological sections were made to observe the morphology of the follicles; blood samples were collected to monitor the concentration of sex hormones (oestradiol and follicle-stimulating hormone). The estrous cycles of the model group mice were disrupted throughout the experiment. Mice in the hUCMSC group and the positive control group resumed normal estrous cycles. The ovarian weight of the model group mice continued to decline. The ovarian weight of the hUCMSC group mice and the positive control group mice decreased first and then gradually increased, and the ovarian weight of the hUCMSC group mice was heavier than that of the positive control group mice. The difference was statistically significant (P<0.05). Compared with the negative control group, the model group experienced a decrease in oestradiol and an increase in follicle-stimulating hormone, and the difference was statistically significant (P<0.05). Compared with the model group, the hUCMSC and positive control groups experienced a slight increase in oestradiol and a decrease in follicle-stimulating hormone; the difference was statistically significant (P<0.05). The pathological examination revealed that the mouse ovaries from the model group were atrophied, the volume was reduced, the cortical and medullary structures were disordered, the number of follicles at all stages was significantly reduced, the number of atretic follicles increased, the number of primordial follicles and corpus luteum significantly decreased, and the corpus luteum had an irregular shape. Compared with those of the model group, the lesions of the hUCMSC and positive control groups significantly improved. The authors concluded that hUCMSCs can repair ovarian tissue damaged by chemotherapy to a certain extent, can improve the degree of apoptosis in ovarian tissue, and can improve the endocrine function of mouse ovaries [2].

In another chemotherapy induced model, POI mice model was generated by cyclophosphamide and busulfan, followed with the treatment of tail-vein injection of the human umbilical cord mesenchymal stem cells (hUCMSCs). Maternal physiological changes and offspring behavior were detected. To reveal the pathogenesis and therapeutic mechanisms of POI, we first compared the metabolite profiles of healthy and POI ovarian tissues using untargeted metabolomics analyses. After stem cell therapy, we then collected the ovaries from control. POI, and hUCMSC-treated POI groups for lipid metabolomics and pseudotargeted metabolomics analysis. Our results revealed remarkable changes of multiple metabolites, especially lipids, in ovarian tissues after POI generation. Following the transplantation of clinical-grade hUCMSCs, POI mice exhibited significant improvements in body weight, sex hormone levels, estrous cycles, and reproductive capacity. Lipid metabolomics and pseudotargeted metabolomics analyses for the ovaries showed that the metabolite levels in the POI group, mainly lipids, glycerophospholipids, steroids, and amino acids changed significantly compared with the controls', and most of them returned to near-healthy levels after receiving hUCMSC treatment. Meanwhile, we also observed an increase of monosaccharide levels in the ovaries from POI mice and a decrease after stem cell treatment. hUCMSCs restore ovarian function through activating the PI3K pathway by promoting the level of free amino acids, consequently improving lipid metabolism and reducing the concentration of monosaccharides. These findings provide potential targets for the clinical diagnosis and treatment of POI [3]. Another chemotherapy associated study, human umbilical cord-MSCs (Huc-MSCs) were assessed for ability to restore fertility through the rescue of ovarian function and reconstruction of the fallopian tubes and uterus. Two mouse models were generated: aging mice (35 weeks of age) and mice with chemotherapy-induced damage. The effect of MSCs on the ovaries, fallopian tubes and uterus was evaluated by analyzing gonadal hormone levels and by performing morphological and statistical analyses. The levels of estradiol (E2) and follicle-stimulating hormone (FSH) exhibited significant recovery after Huc-MSC transplantation in both aging mice and chemotherapy-treated mice. Huc-MSC treatment also increased the number of primordial, developing and preovulatory follicles in the ovaries of mice. Moreover, MSCs were shown to rescue the morphology of the fallopian tubes and uterus through mechanisms such as cilia regeneration in the fallopian tubes and reformation of glands and endometrial tissue in the uterus. The authors concluded that MSCs may represent an effective treatment for restoring female fertility through recovery from chemotherapy-induced damage and rescue of female reproductive organs from the effects of aging [4]. Protection from ovarian failure by umbilical cord MSC was not restricted to chemotherapy induced injury. Wang et al evaluated these cells in an autoimmune model, designed to determine the role of UCMSCs in immune factor-induced POF in rats. In this study, different concentrations of UCMSCs were injected into induced POF rats. Ovarian functions were examined by evaluating the estrus cycle, follicular morphology, hormonal secretion, and the proliferation and apoptosis of granulosa cells. The results showed that the estrus cycle of rats returned to normal and follicular development was significantly improved after transplantation of UCMSCs. In addition, serum concentrations of 17-estradiol (E2), progesterone (P4), and anti-Müllerian hormone (AMH) increased significantly with treatment. Transplantation of UCMSCs also reduced the apoptosis of granulosa cells and promoted the proliferation of granulosa cells. All of these improvements were dose dependent. Furthermore, the results of related gene expression showed that transplanted human UCMSCs upregulated the expression of Bcl-2. AMH, and FSHR in the ovary of POF rats and downregulated the expression of caspase-3. These results further validated the potential mechanisms of promoting the release of cell growth factors and enhancing tissue regeneration and provide a theoretical basis for the clinical application of stem cells in the treatment of premature ovarian failure [5]. In another autoimmune model. 80 female SD rats aged between 6 and 8 weeks were randomly divided into 4 groups A, B. C and D. Rats in group A is normal control group; group B. C and D received zona pellucida glycoprotein 3 (pZP3) administration to induce POF model. Among these, group B is model control group; group C received PBS injection in ovaries and group D received hUCMSCs injection in ovaries, all injections were performed after modeling on the same day. Estrus cycle; serum hormone level of luteinizing hormone (LH) and follicle-stimulating hormone (FSH) and number of ovarian follicles were detected 20 days after treatment. The authors successfully injected hUCMSCs in the ovary tissue of a POF rat. The estrus cycle and hormone expression of the rats in group D tends to be normal. Histological studies indicated that hUCMSCs transplantation increased the number of ovarian follicles. It was concluded that hUCMSCs have a preventive effect on POF rats [6]. Yang et al used a collagen scaffold loaded with human umbilical cord-derived mesenchymal stem cells (collagen/UC-MSCs) transplantation in POF mice preserved ovarian function, as supported by increased estrogen (E₂) and anti-Mullerian hormone (AMH) levels, increased ovarian volume, and an increased number of antral follicles. Immunohistochemistry results of Ki67 indicated transplantation of collagen/UC-MSCs promoted granulosa cell proliferation, which is crucial to oocyte maturation and follicular development. Additionally, transplantation of collagen/UC-MSCs significantly promoted ovarian angiogenesis with the increased expression of CD31. In general, collagen/UC-MSCs transplantation probably is an effective therapeutic strategy of POF [7]. In another study, scientists aimed to fabricate a Matrigel scaffold loaded with human umbilical cord-derived mesenchymal stem cells (MSCs) and explore its potential to restore ovarian function and repair ovarian structures in vitro and in vivo. POF mouse models were established by injecting mice with cyclophosphamide for 15 consecutive days. Then. MSC/Matrigel was transplanted into the ovaries of the mice. Five weeks later, the morphology of the ovaries and follicles was observed by hematoxylin/eosin staining, and the tissue fibrosis ratio was measured using Masson's trichrome staining. The number of blood vessels was evaluated by α-smooth muscle actin and CD31 immunofluorescence, and Ki67 expression was used to determine the proliferation of granulosa cells. The expression of vascular endothelial growth factor (VEGF)-A was assessed by western blotting. The Matrigel scaffold regulated the expression of VEGF-A in vitro. Moreover, it promoted MSC survival and proliferation and prevented MSC apoptosis in vivo. After the transplantation of the MSC/Matrigel, the number of follicles was significantly increased in the mice with POF, and the tissue fibrosis ratio was reduced. Furthermore, the MSC/Matrigel significantly improved the proliferation rate of granulosa cells, increased the number of blood vessels, and upregulated the expression of VEGF-A. These findings demonstrate that MSC/Matrigel may support follicular development and help restore ovarian structures in vivo [8]. In a study to evaluate mechanisms of action. Zheng et al. evaluated the effects of human umbilical cord mesenchymal stem cell (UCMSC) transplantation on ovarian function after ovarian damage caused by chemotherapy and the mechanism underlying these effects were investigated. POF model rats were obtained by the intraperitoneal injection of cyclophosphamide, and cultured UCMSCs were transplanted by tail vein injection. Serum estrogen, follicle-stimulating hormone, gonadotropin releasing hormone, and anti-Mullerian hormone levels were detected by ELISA. Folliculogenesis was evaluated by histopathological examination. The expression levels of nerve growth factor (NGF), high affinity nerve growth factor receptor (TrkA), follicle-stimulating hormone receptor (FSHR), and caspase-3 were evaluated by western blotting and RT-qPCR. The natural reproductive capacity was assessed by pregnant rate and numbers of embryos. The results indicated that UCMSC transplantation recovered disturbed hormone secretion and folliculogenesis in POF rats. NGF and TrkA levels increased, while FSHR and caspase-3 decreased. The pregnancy rate of POF rats was improved. Therefore, UCMSCs could reduce ovarian failure due to premature senescence caused by chemotherapy, and the NGF/TrkA signaling pathway was involved in the amelioration of POF [9]. Another mechanistic experiment involved 18 month-old C57 mice that were randomly divided into a model group and a treatment group. At the same time. 2-month-old C57 mice were established as a young group (15 mice per group). The mice in the treatment group were injected via the tail vein with GFP-labelled mUCMSCs. The ovarian volume in ageing C57 mice was decreased, and there were no follicles at any stage. After mUCMSC transplantation, the mouse ovaries increased in size, follicles at various stages were observed in the cortex, and the antral follicle counts increased. The serum E2, AMH, and INH-B levels of mice in the treatment group were significantly higher than those of mice in the model control group (P<0.05). mUCMSCs downregulated the expression of the autophagy-related gene LC3b and the apoptosis-related genes Bax and Caspase-3, upregulated the expression of SOD2 and the peroxidase gene PRDX IV, and reduced apoptosis rates and reactive oxygen species (ROS) levels in granulosa cells. It was concluded that mUCMSCs play roles in promoting the repair of ageing ovaries by regulating immunity, anti-inflammatory responses and the PI3K-Akt signalling pathway [10]. Another mechanistic study evaluated changes to the immune system. Lu et al. The POF model was established in mice treated with zona pellucida 3 polypeptide fragment (zona pellucida 3. ZP3). The hUMSCs were transplanted into the POF mice through tail vein injection. Following the transplantation, the serum hormone levels of follicle stimulating hormone (FSH), estrogen (E₂), progesterone (P), γ-interferon (IFN-γ), interleukin-2 (IL-2), and interleukin-4 (IL-4) were evaluated by ELISA analysis. Morphological changes of ovarian and uterus tissues were examined by HE staining and immunohistochemistry. The expression of Th1/Th2 cytokines of T cells in spleen and CD56⁺CD16⁻ cells (uterine natural killer cells, uNK cells) in uterine was measured by flow cytometry (FCM) and immunohistochemistry. The expression of HOXA10 in uterine endometrium was examined by immunohistochemistry and RT-PCR analysis. The pinopodes of epithelial cells in uterine endometrium were examined by scanning electron microscopy. Following hUC-MSC transplantation, the serum levels of E₂. P, and IL-4 were increased but FSH, IFN-γ, and IL-2 levels were decreased in POF mice. Also, the transplantation of hUC-MSCs caused an increase in total number of healthy follicles and decrease of atresia follicles. The expression of HOXA10 gene was significantly increased but the CD56⁺CD16⁻ uNK cells decreased in the endometrium of uterine. The ratio of Th1/Th2 cytokines was also significantly decreased. The data suggest that the recovery of ovarian function and endometrial receptivity in POF mice was regulated by the balance of Th1/Th2 cytokines and expression of uNK cells in the endometrium following hUC-MSC transplantation [11]. Another mechanism of action study chemotherapy-induced POF models were induced by intraperitoneal injection of cyclophosphamide. Ovarian function parameters, granulosa cell (GC) apoptosis, and inflammation were examined. Morphological staining showed that UC-MSC treatment increased the ovary size, and the numbers of primary and secondary follicles, but decreased the number of atretic follicles. Estradiol levels in the UC-MSC-treated group were increased while follicle-stimulating hormone levels were reduced compared to those in the POF group. hUC-MSCs inhibited cyclophosphamide-induced GC apoptosis and inflammation. Meanwhile, phosphorylation of AKT and P38 was elevated after UC-MSC treatment. Tracking of hUC-MSCs in vivo indicated that transplanted UC-MSCs were only located in the interstitium of ovaries rather than in follicles. Importantly. UC-MSC-derived extracellular vesicles protected GCs from alkylating agent-induced apoptosis and inflammation in vitro. The results suggest that UC-MSC transplantation can reduce ovary injury and improve ovarian function in chemotherapy-induced POF mice through anti-apoptotic and anti-inflammatory effects via a paracrine mechanism [12]. One important question is whether we can increase efficacy of stem cells by administering multiple injections. In a study, Female mice were intraperitoneally injected with 30 mg/kg busulfan and 120 mg/kg cyclophosphamide (CTX) to induce POF. In the single hUC-MSC injection group, hUC-MSCs were transplanted into mice D7 after CTX and busulfan administration, while in the multiple injection group, hUC-MSCs were transplanted on D7. D14, and D21 after CTX and busulfan administration. We evaluated the ovarian morphology, fertility, follicle-stimulating hormone and estradiol concentrations, follicle count. POF model, and cell transplantation results. In addition, real-time polymerase chain reaction, immunohistochemistry, and miRNA and mRNA chips were used to evaluate the effect of the cell therapy. Ovary size, number of follicle at all developmental stages, and fertility were significantly reduced in the POF group compared with the control. Under hUC-MSC treatment, the ovarian morphology and follicle count were significantly restored, and fertility was significantly increased. By comparing the single and multiple hUC-MSC injection groups, we found that the anti-Müllerian hormone and Ki-67 levels were significantly increased in the multiple hUC-MSC group on D60 after chemotherapy. The expression of stimulating hormone receptors, inhibin α, and inhibin β was significantly restored, and the therapeutic effect was superior to that of the single hUC-MSC injection group. These results indicate that hUC-MSCs can restore the structure of injured ovarian tissue and its function in chemotherapy-induced POF mice and ameliorate fertility. Multiple hUC-MSC transplantations have a better effect on the recovery of ovarian function than single hUC-MSC transplantation in POF [13]. In another effort to increase efficacy of umbilical cord mesenchymal stem cells, a study was conducted to determine whether human umbilical cord blood platelet-rich plasma (ucPRP) enhances the beneficial effects of hUC-MSCs in the treatment of POF. First, we observed the effects of changes in the biological activity of ucPRP on hUC-MSCs in vitro. Subsequently, we tracked the distribution and function of the hUC-MSCs in POF rats, and the rats' estrus cycle and serum sex hormones, follicular development, ovarian angiogenesis, ovarian granulosa cell proliferation, and apoptosis were assessed. The results of the study showed that the addition of ucPRP in vitro accelerates proliferation and reduces apoptosis of the hUC-MSCs while upregulating the stemness gene of the hUC-MSCs. The combined transplantation of hUC-MSCs and ucPRP resulted in more stem cells being retained in the ovaries of POF rats, the estrus cycle of the POF rats being restored, the levels of serum E2, AMH, and FSH improving, and damaged follicles beginning to grow. Finally, we confirmed that the potential mechanism of the combination of hUC-MSCs and ucPRP to rescue the ovarian function of POF rats is to promote ovarian angiogenesis and to promote the proliferation and reduce the apoptosis of ovarian granulosa cells. The upregulation of AMH and FHSR expression and the downregulation of caspase-3 expression in granulosa cells are potential mechanisms for the recovery of ovarian function. Our research results suggest that the combined application of hUC-MSCs and ucPRP is a safe and efficient transplantation program for the treatment of POF, thus providing a reliable experimental basis for the clinical application of stem cell therapy in POF [14].

### SUMMARY

Preferred embodiments are directed to methods of inhibiting ovarian failure and/or stimulating ovarian repair/regeneration by administration of a T cell population expressing FoxP3.

Preferred methods are directed to embodiments wherein said T cell population expresses TGF-beta protein in a membrane bound form.

Preferred methods are directed to embodiments wherein said T cell population expresses GITR and/or GITR ligand.

Preferred methods are directed to embodiments wherein said T cell population possesses ability to suppress a mixed lymphocyte reaction.

Preferred methods are directed to embodiments wherein said suppression of said mixed lymphocyte reaction is inhibition of cytokine production.

Preferred methods are directed to embodiments wherein said suppression of said mixed lymphocyte reaction is inhibition of interferon gamma production.

Preferred methods are directed to embodiments wherein said suppression of said mixed lymphocyte reaction is inhibition of interleukin-2 production.

Preferred methods are directed to embodiments wherein said suppression of said mixed lymphocyte reaction is inhibition of interleukin-9 production.

Preferred methods are directed to embodiments wherein said suppression of said mixed lymphocyte reaction is inhibition of interleukin-12 production.

Preferred methods are directed to embodiments wherein said suppression of said mixed lymphocyte reaction is inhibition of interleukin-15 production.

Preferred methods are directed to embodiments wherein said suppression of said mixed lymphocyte reaction is inhibition of interleukin-15 production.

Preferred methods are directed to embodiments wherein said Foxp3 expressing cells express interleukin-3 receptor.

Preferred methods are directed to embodiments wherein said Foxp3 expressing cells express TGF-beta.

Preferred methods are directed to embodiments wherein said Foxp3 expressing cells express Helios.

Preferred methods are directed to embodiments wherein said Foxp3 expressing cells express CD25.

Preferred methods are directed to embodiments wherein said Foxp3 expressing cells express interleukin-10.

Preferred methods are directed to embodiments wherein said Foxp3 expressing cells express CTLA-4.

Preferred methods are directed to embodiments wherein said Foxp3 expressing cells express CD105.

Preferred methods are directed to embodiments wherein said Foxp3 expressing cells express CD90.

Preferred methods are directed to embodiments wherein said Foxp3 expressing cells express c-kit.

Preferred methods are directed to embodiments wherein said Foxp3 expressing cells express FGF-2.

Preferred methods are directed to embodiments wherein said Foxp3 expressing cells express OCT-2.

Preferred methods are directed to embodiments wherein said Foxp3 expressing cells express KLF.

Preferred methods are directed to embodiments wherein said Foxp3 expressing cells express SOX-2.

Preferred methods are directed to embodiments wherein said Foxp3 expressing cells express NANOG.

Preferred methods are directed to embodiments wherein said FoxP3 expressing cells are pretreated with an activator of indolamine 2-3 deoxygenase.

Preferred methods are directed to embodiments wherein said activator of indolamine 2-3 deoxygenase is interferon alpha.

Preferred methods are directed to embodiments wherein said activator of indolamine 2-3 deoxygenase is interferon gamma.

Preferred methods are directed to embodiments wherein said activator of indolamine 2-3 deoxygenase is interferon beta.

Preferred methods are directed to embodiments wherein said activator of indolamine 2-3 deoxygenase is interleukin-33.

Preferred methods are directed to embodiments wherein ovarian failure is caused by chemotherapy or radiation therapy.

Preferred methods are directed to embodiments wherein said ovarian failure is caused by age.

Preferred methods are directed to embodiments wherein said ovarian failure is ovarian fibrosis.

Preferred methods are directed to embodiments where said ovarian failure is improved by mesenchymal cells.

### DETAILED DESCRIPTION OF THE INVENTION

The invention provides novel means of treatment ovarian failure by administration of T cells expressing FoxP3 alone or in combination with regenerative cells. In one embodiment the T cells are T regulatory cells and they are used, according to the invention, for enhancing therapeutic activities of mesenchymal stem cells by administration of T cells. In specific embodiments the T cells administered are T regulatory cells.

For the purposes of promoting an understanding of the principles of the novel technology, reference will now be made to the preferred embodiments thereof, and specific language will be used to describe the same. It will nevertheless be understood that no limitation of the scope of the novel technology is thereby intended, such alterations, modifications, and further applications of the principles of the novel technology being contemplated as would normally occur to one skilled in the art to which the novel technology relates are within the scope of this disclosure and the claims.

As used herein, unless explicitly stated otherwise or clearly implied otherwise the term ‘about’ refers to a range of values plus or minus 10 percent, e.g. about 1.0 encompasses values from 0.9 to 1.1.

As used herein, unless explicitly stated otherwise or clearly implied otherwise the terms ‘therapeutically effective dose,’ ‘therapeutically effective amounts,’ and the like, refers to a portion of a compound that has a net positive effect on the health and wellbeing of a human or other animal. Therapeutic effects may include an improvement in longevity, quality of life and the like these effects also may also include a reduced susceptibility to developing disease or deteriorating health or wellbeing. The effects may be immediate realized after a single dose and/or treatment or they may be cumulative realized after a series of doses and/or treatments.

The invention teaches the use of T regulatory cells to prevent, inhibit or reverse ovarian failure. In one embodiment, the invention provides for administration of exogenous T regulatory cells in a patient at risk of ovarian failure or suffering from ovarian failure. In another embodiment, the invention provides the use of agents which augment activity and/or number of endogenous T regulatory cells.

In some embodiments of the invention, stimulation of T regulatory cells in vivo is accomplished by administration of Aldesleukin (Proleukin, Novartis), which is a commercially available IL-2 licensed for the treatment of metastatic renal cell carcinoma in the UK. It is produced by recombinant DNA technology using an Escherichia coli strain, which contains a genetically engineered modification of the human IL-2 gene, and is administered either intravenously or subcutaneously (SC). Following short intervenous infusion, its pharmacokinetic profile is typified by high plasma concentrations, rapid distribution into the extravascular space and a rapid renal clearance. The recommended doses for continuous infusion and subcutaneous injection (as detailed in the Summary of Product Characteristics) are repeated cycles of 18×10⁶ IU per m² per 24 hours for 5 days and repeated doses of 18×10⁶ IU, respectively. Peak plasma levels are reached in 2-6 hours after SC administration, with bioavailability of aldesleukin ranging between 31% and 47%. The process of absorption and elimination of subcutaneous aldesleukin is described by a one-compartment model, with a 45 min absorption half-life and an elimination half-life of 3-5 hours [15]. Natural IL-2 was first identified in 1976 as a growth factor for T lymphocytes. It is produced by human cluster designation (CD) 4+ and some CD8+ T-cells and is synthesized mainly by activated T-cells, in particular CD4.sup.+ helper T cells. It stimulates the proliferation and differentiation of T cells, induces the generation of cytotoxic T lymphocytes (CTLs) and the differentiation of peripheral blood lymphocytes to cytotoxic cells and lymphokine-activated killer (LAK) cells, promotes cytokine and cytolytic molecule expression by T cells, facilit:ites the proliferation and differentiation of B-cells and the synthesis of immunoglobulin by B-cells, and stimulates the generation, proliferation and activation of natural killer (NK). IL-2 is known to play a central role in the generation of immune responses. In cancer clinical trials, high-dose recombinant IL-2 (e.g., IV bolus dose of 600,000 international units (IU)/kg every 8 hours for up to 14 doses) demonstrated antitumor activity in metastatic renal cell carcinoma (RCC) and metastatic melanoma. Accordingly, such high-dose IL-2 was approved for the treatment of metastatic RCC in Europe in 1989 and in the US in 1992. In 1998, approval was obtained to treat patients with metastatic melanoma. Recombinant human IL-2 (Aldesleukin) (Proleukin®-Novartis Inc. & Prometheus Labs Inc.) is currently approved by the United States Food and Drug Administration (US FDA). However, IL-2 has a dual function in the immune response in that it not only mediates expansion and activity of effector cells, but also is crucially involved in maintaining peripheral immune tolerance. A major mechanism underlying peripheral self-tolerance is IL-2 induced activation-induced cell death (AICD) in T cells. AICD is a process by which fully activated T cells undergo programmed cell death through engagement of cell surface-expressed death receptors such as CD95 (also known as Fas) or the TNF receptor. When antigen-activated T cells expressing a high-affinity IL-2 receptor (after previous exposure to IL-2) during proliferation are re-stimulated with antigen via the T cell receptor (TCR)/CD 3 complex, the expression of Fas ligand (FasL) and/or tumor necrosis factor (TNF) is induced, making the cells susceptible for Fas-mediated apoptosis. This process is IL-2 dependent and mediated via STAT5. By the process of AICD in T lymphocytes tolerance can not only be established to self-antigens, but also to persistent antigens that are clearly not part of the host's makeup, such as tumor antigens.

In some embodiments of the invention, administration of angiogenic genes is performed in the ovarian to enhance efficacy of Treg cell therapy. Genes with angiogenic ability include: activin A, adrenomedullin, aFGF, ALK1, ALK5, ANF, angiogenin, angiopoietin-1, angiopoietin-2, angiopoietin-3, angiopoietin-4, bFGF, B61, bFGF inducing activity, cadherins, CAM-RF, cGMP analogs, ChDI, CLAF, claudins, collagen, connexins, Cox-2, ECDGF (endothelial cell-derived growth factor), ECG, ECI, EDM, EGF, EMAP, endoglin, endothelins, endostatin, endothelial cell growth inhibitor, endothelial cell-viability maintaining factor, endothelial differentiation shingoingolipid G-protein coupled receptor-1 (EDG1), ephrins, Epo, HGF, TGF-beta, PD-ECGF, PDGF, IGF, IL8, growth hormone, fibrin fragment E, FGF-5, fibronectin, fibronectin receptor, Factor X, HB-EGF, HBNF, HGF, HUAF, heart derived inhibitor of vascular cell proliferation, IL1, IGF-2 IFN-gamma, α1β1 integrin, α2β1 integrin, K-FGF, LIF, leiomyoma-derived growth factor, MCP-1, macrophage-derived growth factor, monocyte-derived growth factor, MD-ECI, MECIF, MMP2, MMP3, MMP9, urokinase plasminogen activator, neuropilin, neurothelin, nitric oxide donors, nitric oxide synthases (NOSs), notch, occludins, zona occludins, oncostatin M, PDGF, PDGF-B, PDGF receptors, PDGFR-β, PD-ECGF, PAI-2, PD-ECGF, PF4, P1GF, PKR1, PKR2, PPAR-gamma, PPAR-gamma ligands, phosphodiesterase, prolactin, prostacyclin, protein S, smooth muscle cell-derived growth factor, smooth muscle cell-derived migration factor, sphingosine-1-phosphate-1 (SIP1), Syk, SLP76, tachykinins, TGF-beta, Tie 1, Tie2, TGF-β, TGF-β receptors, TIMPs, TNF-α, transferrin, thrombospondin, urokinase, VEGF-A, VEGF-B, VEGF-C, VEGF-D, VEGF-E, VEGF, VEGF(164), VEGI, and EG-VEGF.

In one embodiment of the invention, patients suffering from ovarian failure are pretreated with 0.3×10⁶ IU of aldesleukin daily. Concentrations for clinical uses of aldesleukin could be used from the literature as described for other indications including heart failure [15], Wiskott-Aldrich syndrome [16], Graft Versus Host Disease [17, 18], lupus [19], type 1 diabetes [20-22] and are incorporated by reference. In some embodiments of the invention, administration of low doses of IL-2 in the form of aldesleukin every day at concentrations of 0.3×10⁶ to 3.0×10⁶ IU IL-2 per square meter of body surface area for 8 weeks, or in other embodiments repetitive 5-day courses of 1.0×10⁶ to 3.0×10⁶ IU IL-2. Various types of IL-2 may be utilized. Examples of IL-2 variants, recombinant IL-2, methods of IL-2 production, methods of IL-2 purification, methods of formulation, and the like are well known in the art and can be found, for example, at least in U.S. Pat. Nos. 4,530,787, 4,569,790, 4,572,798, 4,604,377, 4,748,234, 4,853,332, 4,959,314, 5,464,939, 5,229,109, 7,514,073, and 7,569,215, each of which is herein incorporated by reference in their entirety for all purposes. In some embodiments, low dose interleukin-2 is provided together with activators of coinhibitory molecules, otherwise known as checkpoints. Such coinhibitory molecules include CTLA-4, PD-1, VISTA, B7-H2, B7-H3, PD-L1, B7-H4, B7-H6, ICOS, HVEM, PD-L2, CD160, gp49B, PIR-B, KIR family receptors, TIM-1, TIM-3, TIM-4, LAG-3, GITR, 4-IBB, OX-40, BTLA, SIRPalpha (CD47), CD48, 2B4 (CD244), B7.1, B7.2, ILT-2, ILT-4, TIGIT, HHLA2, butyrophilins, A2aR, and combinations thereof. In some embodiments of the invention, mesenchymal stem cells are co-administered. Protocols for use of MSC have been previously published and incorporated by reference [23, 24]. For example, mesenchymal stem cells of adipose [25-28], bone marrow [29-48], placental [49], amniotic membrane [50, 51], umbilical cord [52-58], menstrual blood [59], and ovarian [60, 61], origin, as well as conditioned media [62-69]. Additionally, the generation of Treg by mesenchymal stem cells is also described in the art, for which we are providing the following references to assist in the practice of the invention [70-98].

In other embodiments, patients with ovarian failure are administered human IL-2 muteins that preferentially stimulate T regulatory (Treg) cells. As used herein “preferentially stimulates T regulatory cells” means the mutein promotes the proliferation, survival, activation and/or function of CD3+FoxP3+ T cells over CD3+FoxP3+ T cells. Methods of measuring the ability to preferentially stimulate Tregs can be measured by flow cytometry of peripheral blood leukocytes, in which there is an observed increase in the percentage of FOXP3+CD4+ T cells among total CD4+ T cells, an increase in percentage of FOXP3+CD8+ T cells among total CD8+ T cells, an increase in percentage of FOXP3+ T cells relative to NK cells, and/or a greater increase in the expression level of CD25 on the surface of FOXP3+ T cells relative to the increase of CD25 expression on other T cells. Preferential growth of Treg cells can also be detected as increased representation of demethylated FOXP3 promoter DNA (i.e. the Treg-specific demethylated region, or TSDR) relative to demethylated CD3 genes in DNA extracted from whole blood, as detected by sequencing of polymerase chain reaction (PCR) products from bisulfite-treated genomic DNA. IL-2 muteins that preferentially stimulate Treg cells increase the ratio of CD3+FoxP3+ T cells over CD3+FoxP3+ T cells in a subject or a peripheral blood sample at least 30%, at least 40%, at least 50%, at least 60%, at least 70%, at least 80%, at least 90%, at least 100%, at least 150%, at least 200%, at least 300%, at least 400%, at least 500%, at least 600%, at least 700%, at least 800%, at least 900%, or at least 1000%.

In some embodiments of the invention, patients suffering from ovarian failure are administered mesenchymal stem cells together with a tolerance inducing agent, said “agent” is meant to encompass essentially any type of molecule that can be used as a therapeutic properties to enhance T regulatory stimulating capable of mesenchymal stem cells administered in an allogeneic host. Proteins, such as antibodies, fusion proteins, and soluble ligands, any of which may either be identical to a wild-type protein or contain a mutation (i.e., a deletion, addition, or substitution of one or more amino acid residues), and the nucleic acid molecules that encode them (or that are “antisense” to them; e.g., an oligonucleotide that is antisense to the nucleic acids that encode a target polypeptide, or a component (e.g., a subunit) of their receptors), are all “agents.” The agents of the invention can either be administered systemically, locally, or by way of cell-based therapies (i.e., an agent of the invention can be administered to a patient by administering a cell that expresses that agent to the patient). A tolerance restoring agent can be .alpha.1-antitrypsin (AAT; sometimes abbreviated A1AT), which is also referred to as .alpha.1-proteinase inhibitor. AAT is a major serum serine-protease inhibitor that inhibits the enzymatic activity of numerous serine proteases including neutrophil elastase, cathespin G, proteinase 3, thrombin, trypsin and chymotrypsin. For example, one can administer an AAT polypeptide (e.g., a purified or recombinant AAT, such as human AAT) or a homolog, biologically active fragment, or other active mutant thereof. .alpha.1 proteinase inhibitors are commercially available for the treatment of AAT deficiencies, and include ARALAST™, PROLASTIN™, and ZEMAIRA™. The AAT polypeptide or the biologically active fragment or mutant thereof can be of human origin and can be purified from human tissue or plasma. Alternatively, it can be recombinantly produced. For ease of reading, we do not repeat the phrase “or a biologically active fragment or mutant thereof” after each reference to AAT. It is to be understood that, whenever a full-length, naturally occurring AAT can be used, a biologically active fragment or other biologically active mutant thereof (e.g., a mutant in which one or more amino acid residues have be substituted) can also be used. Similarly, we do not repeat on each occasion that a naturally occurring polypeptide (e.g., AAT) can be purified from a natural source or recombinantly produced. It is to be understood that both forms may be useful. Similarly, we do not repeatedly specify that the polypeptide can be of human or non-human origin. While there may be advantages to administering a human protein, the invention is not so limited.

The methods of the present invention (e.g., multiple-variable dose IL-2 alone or in combination with one or more other anti-immune disorder therapies) can be administered to a desired subject or once a subject is indicated as being a likely responder to such therapy. In another embodiment, the therapeutic methods of the present invention can be avoided if a subject is indicated as not being a likely responder to the therapy and an alternative treatment regimen, such as targeted and/or untargeted anti-immune therapies, can be administered.

In one embodiment, a multiple-variable IL-2 dose method of treating a subject suffering from ovarian failure a therapy comprising a) administering to the subject an induction regimen comprising continuously administering to the subject interleukin-2 (IL-2) at a dose that increases the subject's plasma IL-2 level and increases the subject's ratio of immune suppressive T cells to conventional T lymphocytes (Tcons) and b) subsequently administering to the subject at least one maintenance regimen comprising continuously administering to the subject an IL-2 maintenance dose that is higher than the induction regimen dose and that i) further increases the subject's plasma IL-2 level and ii) further increases the ratio of immune suppressive T cells to Tcons, thereby treating the subject, is provided. In one embodiment, the level of plasma IL-2 resulting from the induction regimen is depleted below that of the prior peak plasma IL-2 level before the induction regimen. The IL-2 maintenance regimen can, in certain embodiments, increase the subject's plasma IL-2 level beyond the peak plasma IL-2 level induced by the induction regimen. The term “multiple-variable IL-2 dose method” refers to a therapeutic intervention comprising more than one IL-2 administration, wherein the more than one IL-2 administration uses more than one IL-2 dose. Such a method is contrasted from a “fixed” dosed method wherein a fixed amount of IL-2 is administered in a scheduled manner, such as daily. The term “induction regimen” refers to the continuous administration of IL-2 at a dose that increases the subject's plasma IL-2 level and increases the subject's immune suppressive T cells:Tcons ratio. In some embodiments, the regimen occurs until a peak level of plasma IL-2 is achieved. The subject's plasma IL-2 level and/or immune suppressive T cell:Tcons ratio can be increased by at least 5%, 10%, 15%, 20%, 25%, 30%, 35%, 40%, 45%, 50%, 55%, 60%, 65%, 70%, 75%, 80%, 85%, 90%, 95%, 100%, 110%, 120%, 130%, 140%, 150%, 160%, 170%, 180%, 190%, 200% or more relative to the baseline ratio prior to initiation of therapy.

In one embodiment of the invention certain doses and methods according to FDA-approved uses, Tcons are preferentially activated relative to immune suppressive T cells such that the immune suppressive T cells:Tcons ratio actually decreases. By contrast, the methods of the present invention increase the immune suppressive T cells:Tcons ratio by using “low-dose IL-2” in a range determined herein to preferentially promote immune suppressive T cells over Tcons and that are safe and efficacious in subjects suffering from ovarian failure.

The term “low-dose IL-2” refers to the dosage range wherein immune suppressive T cells are preferentially enhanced relative to Tcons. In one embodiment, low-dose IL-2 refers to IL-2 doses that are less than or equal to 50% of the “high-dose IL-2” doses (e.g., 18 million IU per m.sup.2 per day to 20 million IU per m.sup.2 per day, or more) used for anti-cancer immunotherapy. The upper limit of “low-dose IL-2” can further be limited by treatment adverse events, such as fever, chills, asthenia, and fatigue. IL-2 is generally dosed according to an amount measured in international units (IU) administered in comparison to body surface area (BSA) per given time unit. BSA can be calculated by direct measurement or by any number of well-known methods (e.g., the Dubois & Dubois formula), such as those described in the Examples. Generally, IL-2 is administered according in terms of IU per m.sup.2 of BSA per day. Exemplary low-dose IL-2 doses according to the methods of the present invention include, in terms of 10.sup.6 IU/m.sup.2/day, any one of 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, and 3.0.times.10.sup.6 IU/m.sup.2/day, including any values in between and/or ranges in between. For example, an induction regimen dose can range between 0.3.times.10.sup.6 IU/m.sup.2/day and 3.0.times.10.sup.6 IU/m.sup.2/day with any value or range in between.

The term “continuous administration” refers to administration of IL-2 at regular intervals without any intermittent breaks in between. Thus, no interruptions in IL-2 occur. For example, the induction dose can be administered every day (e.g., once or more per day) during at least 1-14 consecutive days or any range in between (e.g., at least 4-7 consecutive days). As described herein, longer acting IL-2 agents and/or IL-2 agents administered by routes other than subcutaneous administration are contemplated. Intermittent intravenous administration of IL-2 described in the art results in short IL-2 half lives incompatible with increasing plasma IL-2 levels and increasing the immune suppressive T cells:Tcons ratio according to the present invention. However, once-daily subcutaneous IL-2 dosing, continuous IV infusion, long-acting subcutaneous IL-2 formulations, and the like are contemplated for achieving a persistent steady state IL-2 level.

As described above, IL-2 can be administered in a pharmaceutically acceptable formulation and by any suitable administration route, such as by subcutaneous, intravenous, intraperitoneal, oral, nasal, transdermal, or intramuscular administration. In one embodiment, the present invention provides pharmaceutically acceptable compositions which compose IL-2 at a therapeutically-effective amount, formulated together with one or more pharmaceutically acceptable carriers (additives) and/or diluents. The pharmaceutical compositions of the present invention may be specially formulated for administration in solid or liquid form, including those adapted for the following: (1) oral administration, for example, drenches (aqueous or non-aqueous solutions or suspensions), tablets, boluses, powders, granules, pastes; (2) parenteral administration, for example, by subcutaneous, intramuscular or intravenous injection as, for example, a sterile solution or suspension; (3) topical application, for example, as a cream, ointment or spray applied to the skin; (4) intravaginally or intrarectally, for example, as a pessary, cream or foam; or (5) aerosol, for example, as an aqueous aerosol, liposomal preparation or solid particles containing the compound.

In some embodiments of the invention the monoclonal antibody (mAb) against the CD3 molecule is utilized for immune modulation. This approach has previously been used to induced tolerance to autoimmunity in murine models of type 1 diabetes mellitus. Treatment with anti-CD3 mAb reversed diabetes in the NOD mouse and prevented recurrent immune responses toward transplanted syngeneic islets. This was achieved without the need for continuous immune suppression and persisted at a time when T cell numbers were not depleted and were quantitatively normal. Another approach is to induce specific immunological unresponsiveness by administering self-antigens.

For the practice of the invention, it is important to utilize the proper type of anti-CD3 antibody. The natural role of CD3 is to transduce signals in T cells from the T cell receptor into the nucleus of the T cells, usually to activity T cells. In some situations, antibodies to CD3 cause activation of T cells, not suppression. For example, Hirsch et al. investigated the ability of low dose anti-CD3 to enhance an anti-tumor response directed against the malignant murine UV-induced skin tumor. Low dose anti-CD3 administration resulted in enhanced in vitro anti-tumor activity and prevented tumor outgrowth in approximately two-thirds of animals treated at the time of tumor inoculation. Furthermore, these animals displayed lasting tumor-specific immunity. Augmentation of various parameters of immunity was noted. These results suggested that anti-CD3 mAb can be utilized for the enhancement of anti-tumor responses in vivo and may have general application in the treatment of immunodeficiency. They also point to the care that needs to be exercised when manipulating the CD3 pathway, given that the pathway can be both activatory or inhibitory [99]. Activatory signals by crosslinking CD3 are also seen in the tumor infiltrating lymphocyte (TIL) culture systems. It is known that early in the life of the TIL bulk culture, cytotoxicity is non-major histocompatibility complex restricted. Under these culture conditions antitumor cytotoxicity was observed to decline with increasing age of the bulk culture. In addition, TIL became refractory to IL-2-induced expansion. In one study, scientists have used solid-phase anti-CD3 antibodies for TIL activation followed by culture in reduced concentrations of IL-2 to reactivate TIL previously grown in high concentrations of rIL-2. TIL refractory to IL-2 in terms of growth and antitumor cytotoxicity proved sensitive to anti-CD3 activation. The use of solid-phase anti-CD3 was also more effective than high concentrations of IL-2 in the expansion of TIL when used at the start of culture. Finally, TIL could be induced to secrete IL-2 following solid-phase activation with anti-CD3. These data suggest that human TIL are susceptible to activation by signals directed at the CD3 complex of the TIL cell surface [100].

An example of how different CD3 targeting antibodies can elicit different effects is seen in another study, which Davis et al. examined the IgM monoclonal antibody called 38.1, which was distinct from other anti-CD3 mAb, in that it was rapidly modulated from the cell surface in the absence of a secondary antibody. Although 38.1 induced an immediate increase in intracellular free calcium [Ca2+]i by highly purified T cells, it did not induce entry of the cells into the cell cycle in the absence of accessory cells (AC) or a protein kinase C-activating phorbol ester. Treated T cells were markedly inhibited in their capacity to respond to the T cell stimulating mitogen phytohemagluttanin. Inhibition of responsiveness could be overcome by culturing the cells with supplemental antigen presenting cells or the cytokine IL-2. These studies demonstrate that a state of T cell nonresponsiveness can be induced by modulating CD3 with an anti-CD3 mAb in the absence of co-stimulatory signals. A brief increase in [Ca2+]i resulting from mobilization of internal calcium stores appears to be sufficient to induce this state of T cell nonresponsiveness [101].

In some situations, anti-CD3 antibodies have been shown to program T cells towards antigen-specific tolerance. This is illustrated in one example in the work of Anasetti et al. who exposed PBMC to alloantigen for 3-8 d in the presence of anti-CD3 antibodies. They showed no response after restimulation with cells from the original donor but the PBMC remained capable of responding to third-party donors. Antigen-specific nonresponsiveness was induced by both nonmitogenic and mitogenic anti-CD3 antibodies but not by antibodies against CD2, CD4, CD5, CD8, CD18, or CD28. This suggested the unique ability of this protein to modulate programs in the T cells that are antigen specific. Nonresponsiveness induced by anti-CD3 antibody in mixed leukocyte culture was sustained for at least 34 d from initiation of the culture and 26 d after removal of the antibody. Anti-CD3 antibody also induced antigen-specific nonresponsiveness in cytotoxic T cell generation assays. Anti-CD3 antibody did not induce nonresponsiveness in previously primed cells [102].

The use of anti-CD3 antibodies for the practice of the invention requires that the antibodies not only do not result in activation of T cell proliferation and inflammatory cytokine secretion, but also that the T cells actually inhibit inflammation and promote regeneration.

In one embodiment of the invention, anti-CD3 antibody is given 14 days before administration of mesenchymal stem cells In one specific embodiment, said 14-day course of the anti-CD3 monoclonal antibody utilizes the antibody hOKT3γ1(Ala-Ala) administered intravenously (1.42 μg per kilogram of body weight on day 1; 5.67 μg per kilogram on day 2; 11.3 μg per kilogram on day 3; 22.6 μg per kilogram on day 4; and 45.4 μg per kilogram on days 5 through 14); these doses were based on those previously used for treatment of transplant rejection [103] which is incorporated by reference. Other types of anti-CD3 molecules and dosing regimens may be used in the context of ovarian failure therapeutics, said doses may be chosen from examples of utility of anti-CD3 from the literature, as described in the following papers and incorporated by reference: prevention of kidney [104-112], liver [113-115], pancreas [116-118], ovarian [119], and heart [120-124] transplant rejection; prevention of graft versus host disease [125], multiple sclerosis [126], type 1 diabetes [127].

The use of monoclonal antibodies for the practice of the invention must be tempered by the caution that in some cases cytokine storm may be initiated by antibody administration [128, 129]. In some cases this is concentration dependent [130]. Treatment for this can be accomplished by steroid administration or anti-IL6 antibody [131-135].

In some embodiments of the invention administration of PGE1 and/or various natural anti-inflammatory compounds are provided to decrease TNF-alpha production as a result of anti-CD3 administration, such as described in this paper and incorporated by reference [136]. In further embodiments of the invention, administration of anti-CD3 may be performed together with endothelial protectants and/or anti-coagulants in order to reduce clotting associated with CD3 modulating agents [137]. In some embodiments anti-CD3 antibodies may be used in combination with tolerogenic cytokines such as interleukin-10 in order to enhance number of angiogenesis supporting T cells. The safety of anti-CD3 and IL-10 administration has previously been demonstrated in a clinical trial [138].

In the current invention decreased TNF-alpha activity is correlated with enhancement of ovarian recovery. Furthermore, other inhibitors of TNF-alpha may be administered [139, 140].

In some embodiments of the invention, enhancement of ovarian recovery is provided by administration of oral modulators of CD3. Oral administration of OKT3 has been previously performed in a clinical trial and results are incorporated by reference [141, 142].

The phrase “pharmaceutically acceptable” is employed herein to refer to those agents, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio. The phrase “pharmaceutically-acceptable carrier” as used herein means a pharmaceutically-acceptable material, composition or vehicle, such as a liquid or solid filler, diluent, excipient, solvent or encapsulating material, involved in carrying or transporting the subject chemical from one organ, or portion of the body, to another organ, or portion of the body. Each carrier must be “acceptable” in the sense of being compatible with the other ingredients of the formulation and not injurious to the subject. Some examples of materials which can serve as pharmaceutically-acceptable carriers include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) phosphate butler solutions; and (21) other non-toxic compatible substances employed in pharmaceutical formulations. Formulations useful in the methods of the present invention include those suitable for oral, nasal, topical (including buccal and sublingual), rectal, vaginal, aerosol and/or parenteral administration. The formulations may conveniently be presented in unit dosage form and may be prepared by any methods well known in the art of pharmacy. The amount of active ingredient which can be combined with a carrier material to produce a single dosage form will vary depending upon the host being treated, the particular mode of administration. The amount of active ingredient, which can be combined with a carrier material to produce a single dosage form will generally be that amount of the compound which produces a therapeutic effect. Generally, out of one hundred percent, this amount will range from about 1 percent to about ninety-nine percent of active ingredient, preferably from about 5 percent to about 70 percent, most preferably from about 10 percent to about 30 percent.

In one embodiment, the Treg cell surface protein is selected from the group consisting of CD25, GITR, TIGIT, CTLA-4, neuropilin, OX40, LAG3, and combinations thereof, said Tregs are isolated possessing said surfaces proteins from a tissue source, and optionally expanded ex vivo prior to administration to a patient suffering from ovarian failure.

In one embodiment of the invention, mesenchymal stem cell exosomes are administered in order to enhance therapeutic activity of T regulatory cells and/or low dose interleukin-2 therapy. Exosomes are purified from mesenchymal stem cells by obtaining a mesenchymal stem cell conditioned medium, concentrating the mesenchymal stem cell conditioned medium, subjecting the concentrated mesenchymal stem cell conditioned medium to size exclusion chromatography, selecting UV absorbent fractions at 220 nm, and concentrating fractions containing exosomes.

Exosomes, also referred to as “particles” may comprise vesicles or a flattened sphere limited by a lipid bilayer. The particles may comprise diameters of 40-100 nm. The particles may be formed by inward budding of the endosomal membrane. The particles may have a density of about.1.13-1.19 g/ml and may float on sucrose gradients. The particles may be enriched in cholesterol and sphingomyelin, and lipid raft markers such as GM1, GM3, flotillin and the src protein kinase Lyn. The particles may comprise one or more proteins present in mesenchymal stem cells or mesenchymal stem cell conditioned medium (MSC-CM), such as a protein characteristic or specific to the MSC or MSC-CM. They may comprise RNA, for example miRNA. Said particles may possess one or more genes or gene products found in MSCs or medium which is conditioned by culture of MSCs. The particle may comprise molecules secreted by the MSC. Such a particle, and combinations of any of the molecules comprised therein, including in particular proteins or polypeptides, may be used to supplement the activity of, or in place of, the MSCs or medium conditioned by the MSCs for the purpose of for example treating or preventing a disease. Said particle may comprise a cytosolic protein found in cytoskeleton e.g. tubulin, actin and actin-binding proteins, intracellular membrane fusions and transport e.g. annexins and rab proteins, signal transduction proteins e.g. protein kinases, 14-3-3 and heterotrimeric G proteins, metabolic enzymes e.g. peroxidases, pyruvate and lipid kinases, and enolase-1 and the family of tetraspanins e.g. CD9, CD63, CD81 and CD82. In particular, the particle may comprise one or more tetraspanins. The particles may comprise mRNA and/or microRNA. The particle may be used for any of the therapeutic purposes that the MSC or MSC-CM may be put to use.

In one embodiment, MSC exosomes, or particles may be produced by culturing mesenchymal stem cells in a medium to condition it. The mesenchymal stem cells may comprise human umbilical tissue derived cells which possess markers selected from a group comprising of CD90, CD73 and CD105. The medium may comprise DMEM. The DMEM may be such that it does not comprise phenol red. The medium may be supplemented with insulin, transferrin, or selenoprotein (ITS), or any combination thereof. It may comprise FGF2. It may comprise PDGF AB. The concentration of FGF2 may be about 5 ng/ml FGF2. The concentration of PDGF AB may be about 5 ng/ml. The medium may comprise glutamine-penicillin-streptomycin or b-mercaptoethanol, or any combination thereof. The cells may be cultured for about 1, 2, 3, 4, 5, 6, 7, 8, 9, 10 days or more, for example 3 days. The conditioned medium may be obtained by separating the cells from the medium. The conditioned medium may be centrifuged, for example at 500 g. it may be concentrated by filtration through a membrane. The membrane may comprise a >1000 kDa membrame. The conditioned medium may be concentrated about 50 times or more. The conditioned medium may be subject to liquid chromatography such as HPLC. The conditioned medium may be separated by size exclusion. Any size exclusion matrix such as Sepharose may be used. As an example, a TSK Guard column SWXL, 6.times.40 mm or a TSK gel G4000 SWXL, 7.8.times.300 mm may be employed. The eluent buffer may comprise any physiological medium such as saline. It may comprise 20 mM phosphate buffer with 150 mM of NaCl at pH 7.2. The chromatography system may be equilibrated at a flow rate of 0.5 ml/min. The elution mode may be isocratic. UV absorbance at 220 nm may be used to track the progress of elution. Fractions may be examined for dynamic light scattering (DLS) using a quasi-elastic light scattering (QELS) detector. Fractions which are found to exhibit dynamic light scattering may be retained. For example, a fraction which is produced by the general method as described above, and which elutes with a retention time of 11-13 minutes, such as 12 minutes, is found to exhibit dynamic light scattering. The r.sub.h of particles in this peak is about 45-55 nm. Such fractions comprise mesenchymal stem cell particles such as exosomes.

Culture conditioned media may be concentrated by filtering/desalting means known in the art. In one embodiment Amicon filters, or substantially equivalent means, with specific molecular weight cut-offs are utilized, said cut-offs may select for molecular weights higher than 1 kDa to 50 kDa.

The cell culture supernatant may alternatively be concentrated using means known in the art such as solid phase extraction using C18 cartridges (Mini-Spe-ed C18-14%, S.P.E. Limited, Concord ON). Said cartridges are prepared by washing with methanol followed by deionized-distilled water. Up to 100 ml of stem cell or progenitor cell supernatant may be passed through each of these specific cartridges before elution, it is understood of one of skill in the art that larger cartridges may be used. After washing the cartridges material adsorbed is eluted with 3 ml methanol, evaporated under a stream of nitrogen, redissolved in a small volume of methanol, and stored at 4.degree. C.

Before testing the eluate for activity in vitro, the methanol is evaporated under nitrogen and replaced by culture medium. Said C18 cartridges are used to adsorb small hydrophobic molecules from the stem or progenitor cell culture supernatant, and allows for the elimination of salts and other polar contaminants. It may, however be desired to use other adsorption means in order to purify certain compounds from said fibroblast cell supernatant. Said fibroblast concentrated supernatant may be assessed directly for biological activities useful for the practice of this invention, or may be further purified. In one embodiment, said supernatant of fibroblast culture is assessed for ability to stimulate proteoglycan synthesis using an in vitro bioassay. Said in vitro bioassay allows for quantification and knowledge of which molecular weight fraction of supernatant possesses biological activity. Bioassays for testing ability to stimulate proteoglycan synthesis are known in the art. Production of various proteoglycans can be assessed by analysis of protein content using techniques including mass spectrometry, column chromatography, immune based assays such as enzyme linked immunosorbent assay (ELISA), immunohistochemistry, and flow cytometry.

Further purification may be performed using, for example, gel filtration using a Bio-Gel P-2 column with a nominal exclusion limit of 1800 Da (Bio-Rad, Richmond Calif.). Said column may be washed and pre-swelled in 20 mM Tris-HCl buffer, pH 7.2 (Sigma) and degassed by gentle swirling under vacuum. Bio-Gel P-2 material be packed into a 1.5.times.54 cm glass column and equilibrated with 3 column volumes of the same buffer. Amniotic fluid stem cell supernatant concentrates extracted by C18 cartridge may be dissolved in 0.5 ml of 20 mM Tris buffer, pH 7.2 and run through the column. Fractions may be collected from the column and analyzed for biological activity. Other purification, fractionation, and identification means are known to one skilled in the art and include anionic exchange chromatography, gas chromatography, high performance liquid chromatography, nuclear magnetic resonance, and mass spectrometry. Administration of supernatant active fractions may be performed locally or systemically.

In one embodiment ovarian progenitors are administered, together with mesenchymal stem cell exosomes and/or mesenchymal stem cell conditioned media. In one embodiment ovarian progenitor cells are characterized as having high expression of CD47 (CD47.sup.hi) from the pluripotent stem cell population, thereby isolating one or more ovarian progenitor cells. In one embodiment, the method further comprises sorting the population for low CD26 expression (CD26.sup.lo), such that an isolated population of CD47.sup.hi/CD26.sup.lo ovarian progenitor cells is isolated. In another embodiment of this aspect and all other aspects described herein, the at least one differentiation-inducing agent comprises at least one of CHIR 99021, BMP4, KGF, FGF10, and retinoic acid. In one embodiment, the concentration of CHIR 99021 used with the methods of generating primordial ovarian progenitors as described herein comprises at least 0.5·mu·M, at least 1·mu·M, at least 1.5·mu·M, at least 2·mu·M, at least 2.5·mu·M, at least 3·mu·M, at least 3.5·mu·M, at least 4·mu·M, at least 4.5·mu·M, at least 5·mu·M, at least 1004, at least 20·mu·M or more. In another embodiment, the concentration of CHIR 99021 used with the methods of generating primordial ovarian progenitors as described herein comprises a concentration in the range of 1-5·mu·M, 1-10·mu·M, 1-20·mu·M, 2-4·mu·M, 5-20·mu·M, 10-20·mu·M, or any range there between. In another embodiment, the concentration of BMP4 used with the methods of generating primordial ovarian progenitors as described herein comprises at least 1 ng/mL, at least 2 ng/mL, at least 3 ng/mL, at least 4 ng/mL, at least 5 ng/mL, at least 6 ng/mL, at least 7 ng/mL, at least 8 ng/mL, at least 9 ng/mL, at least 10 ng/mL, at least 11 ng/mL, at least 12 ng/mL, at least 13 ng/mL, at least 14 ng/mL, at least 15 ng/mL, at least 20 ng/mL, at least 30 ng/mL, at least 40 ng/mL, at least 50 ng/mL, at least 60 ng/mL, at least 75 ng/mL, at least 100 ng/mL, at least 125 ng/mL, at least 150 ng/mL, at least 200 ng/mL or more. In another embodiment, the concentration of BMP4 used with the methods of generating primordial ovarian progenitors as described herein comprises a concentration in the range of 1-50 ng/mL, 1-25 ng/mL, 1-10 ng/mL, 5-10 ng/mL, 5-15 ng/mL, 5-25 ng/mL, 25-50 ng/mL, 25-75 ng/mL, 25-100 ng/mL, 25-150 ng/mL, 75-125 ng/mL or any range therebetween.

Another embodiment of the invention teaches isolating a ovarian progenitor cell for use with mesenchymal stem cell exosomes, the method comprising: (a) contacting a population of pluripotent cells with a first binding reagent that recognizes CD47 and a second binding reagent that recognizes CD26 to determine the level of expression of CD47 and CD26, and (b) isolating at least one cell with a cell surface phenotype comprising CD47.sup.hi/CD26.sup.lo, thereby isolating a ovarian progenitor cell from the population of pluripotent cells.

In some embodiments of the invention, administration of exogenous ovarian progenitors and/or ovarian granulosa cells is performed together with the T regulatory cells and/or enhancement of T regulatory cells and/or stem cell administration. Multi-potent cells useful for generation of ovarian progenitors include, but are not limited to, embryonic stem cells (ESCs), pluripotent stem cells, induced pluripotent stem cells (iPSCs) or otherwise reprogrammed somatic cells, bone marrow derived cells, peripheral blood-derived cells. The multi-potent cells may be any mammalian multi-potent cell. In some embodiments, the granulosa cell specific reporter includes a fluorescent reporter under regulatory control of an ovarian granulosa cell-specific gene. Ovarian granulosa cell-specific genes include, but are not limited to, forkhead box L2 (Fox12), wingless type MMTV integration site family, member 4 (WNT4), Nr5a1, Dax-1, ATP-binding cassette, subfamily 9 (Abca9), acetyl-Coenzyme A acyltransferase 2 (mitochondrial 3-oxoacyl-Coenzyme A thiolase; Acaa2), actin, alpha 2, smooth muscle, aorta (Acta2), a disintegrin-like and metallopeptidase (reprolysin-like) with thrombosin type 1 motif, 17 (Adamts17), ADAMTS-like 2 (Adamts12), AF4/FMR2 family, member 1 (Aff1), expressed sequence AI314831 (A1314831), Aldo-keto reductase family 1, member C14 (Akr1c14), aldo-keto reductase family 1, Notch2, and member C-like (Akr1c1). Fluorescent reporters include but are not limited to, Discosoma sp. red (DsRed), green fluorescent protein (GFP), yellow fluorescent protein (YFP), and orange fluorescent protein (OFP). In some embodiments, the granulosa cell specific reporter is a non-fluorescent reporter under regulatory control of an ovarian granulosa cell-specific gene. Non-fluorescent reporters include, but are not limited to, luciferase and beta-galactosidase. The granulosa cell specific reporter can be engineered by any methods known in the art. By way of example, but not by limitation, in some embodiments, a granulosa cell specific reporter is engineered by identifying a granulosa cell specific gene promoter, determining a conserved region of the gene promoter, isolating the conserved region from genomic DNA using PCR, and cloning the conserved region into a vector containing a fluorescent marker. Engineering multi-potent cells to contain the granulosa cell specific gene reporter can be accomplished by any method known in the art. By way of example, but not by limitation, in some embodiments, the granulosa cell specific gene reporter is inserted into the multi-potent cells by using electroporation. Other methods for inserting the granulosa cell specific gene reporter include, but are not limited to, viral transduction, cationic liposomal transfection, multi-component lipid based transfection, calcium phosphate, DEAE-dextran, and direct delivery. Multi-potent cells that contain the granulosa cell specific gene reporter can be selected for by any cell selection method known in the art. Examples of methods for cell selection include, but are not limited to, fluorescence activated cell sorting (FACS), differential adhesion, selection of precursor or progenitor cells for clonal expansion, and selection for antibiotic resistance. Populations of multi-potent cells that contain the granulosa cell specific gene reporter are cultured under conditions suitable for differentiation of the mammalian multi-potent cells to granulosa cells or granulosa cell precursors. The multi-potent cells can be induced to differentiate by any methods commonly used in the art. By way of example, but not by limitations, undifferentiated ESCs containing granulosa cell specific gene reporter that are cultured on a mitotically-inactivated mouse embryonic fibroblast (MEF) feeder layer can be induced to differentiate by separating the ESCs from the MEF by differential adhesion and culturing the ESCs with 15% FBS in the absence of LIF on gelatin-coated plates in a monolayer. Other methods for differentiation include, but are not limited to, inducement by embryoid body formation in hanging droplets. In some embodiments, growth factors or activators of signaling pathways for granulosa cell specification are used to direct multi-potent cell to differentiate into granulosa cells or granulosa cell precursors. Growth factors or activators of signaling pathways for granulosa cell specification, include, but are not limited to bFGF or activators of the Notch signaling pathway, e.g., Jagged1 or Jagged2. After inducement of differentiation of the population of multi-potent cells, granulosa cells or granulosa cell precursors are identified and isolated. In some embodiments, the granulosa cells or granulosa cell precursors are identified by the expression of fluorescence under the control of the granulosa cell-specific gene. In some embodiments, the granulosa cells or granulosa cell precursors are isolated into enriched populations of granulosa cells or granulosa cell precursors by FACS, antibody-based immunomagnetic sorting (e.g., magnetic assisted cell sorting (MACS)), differential adhesion, clonal selection and expansion, or antibiotic resistance. In some embodiments, the granulosa cells or granulosa cell precursors are isolated using a cell surface markers(s) selective for or specific to granulosa cells or granulosa cell precursors. Examples of cell surface markers selective for or specific to granulosa cells or granulosa cell precursors include, but are not limited to anti-mullarian hormone receptor, and Notch receptor (Notch2). In some embodiments, enriched populations of granulosa cells or granulosa cell precursors are used to promote the growth and maturation of follicles, follicle-like structures, and/or immature oocytes in ovarian tissue. In some embodiments, ovarian tissue is contacted by an enriched population of granulosa cells or granulosa cell precursors wherein the granulosa cells or granulosa cell precursors promote the growth and maturation of follicles, follicle-like structures, and/or immature oocytes in ovarian tissue. In some embodiments, after contact with the ovarian tissue, the granulosa cells or granulosa cell precursors migrate to follicles, follicle-like structures, and/or immature oocytes or oocyte precursors in ovarian tissue to produce or enrich an ovarian somatic environment that induces maturation of follicles and immature oocytes. In some embodiments, the ovarian tissue is contacted by an enriched population of granulosa cells or granulosa cell precursors in vivo. In some embodiments, in vivo administration includes, but is not limited to, localized injection (e.g., catheter administration or direct intra-ovarian injection), systemic injection, intravenous injection, intrauterine injection, and parenteral administration.

For the practice of the invention, following systemic or local administration of FoxP3 expressing cells, in some embodiments, the ovarian tissue is contacted by an enriched population of granulosa cells or granulosa cell precursors ex vivo. In some embodiments, ex vivo contact includes, but is not limited to, direct injection of ovarian tissue, aggregation with intact or dissociated ovarian tissue, and co-culture with ovarian tissue. In some embodiments, the contacted ex vivo ovarian tissue is cultured and then transplanted or implanted into a subject's ovaries or surrounding tissues. Methods for transplanting or implanting include, but are not limited to, engraftment onto ovary, injection or engraftment of tissue into ovary following ovarian incision, and engraftment into fallopian tube. In some embodiments, the contacted ex vivo ovarian tissue is cultured and then frozen and stored after growth and maturation of the follicle and/or oocyte. The ovarian tissue may be any mammalian ovarian tissue. Mammals from which the ovarian tissue can originate, include, for example, farm animals, such as sheep, pigs, cows, and horses; pet animals, such as dogs and cats; laboratory animals, such as rats, mice, monkeys, and rabbits. In some embodiments, the mammal is a human. In some embodiments, the enriched population of granulosa cells or granulosa cell precursors and the ovarian tissue are autologous. In some embodiments, the enriched population of granulosa cells or granulosa cell precursors and the ovarian tissue are heterologous allogeneic. In some embodiments, the promotion of growth and maturation of follicles, follicle-like structures, and/or immature oocytes or oocyte precursors in ovarian tissue by granulosa cells or granulosa cell precursors is measured by an increase in follicle diameter, increase in granulosa cell number, increase in steroid hormone production, increase in oocyte diameter, or a combination thereof. The diameter of a maturing follicle or oocyte varies from species to species and is identifiable by one skilled in the art since mature follicle sizes for specific species is generally known in the art. By way of example, but not by limitation, in some embodiments, a follicular diameter of a human follicle that is indicative of a mature or maturing follicle is a diameter greater than about 30·mu·m. Alternatively, or additionally, a follicular diameter of a human follicle that is indicative of a mature or maturing follicle is a diameter between about 30·mu·m to 10,000·mu·m, between about 50·mu·m to 5000·mu·m, between about 100·mu·m to 2000·mu·m, between about 200·mu·m to 1000·mu·m, between about 300·mu·m to 900·mu·m, between about 400·mu·m to 800·mu·m, or between about 500·mu·m to 700·mu·m.

By way of example, but not by limitation, in some embodiments, an oocyte diameter of a human oocyte that is indicative of a mature or maturing oocyte is a diameter greater than about 10·mu·m. Alternatively, or additionally, a follicular diameter of a human follicle that is indicative of a mature or maturing oocyte is a diameter between about 10·mu·m to 200·mu·m, or between about 20·mu·m to 175·mu·m, or between about 30·mu·m to 150·mu·m, or between about 40·mu·m to 125·mu·m, or between about 50·mu·m to 100·mu·m, or between about 60·mu·m to 75·mu·m.

In some embodiments, an increase in granulosa cell number in ovarian tissue is measured by comparison of the number of granulosa cells in the ovarian tissue before contact with granulosa cells or granulosa cell precursors to the number of granulosa cells in the ovarian tissue after contact with granulosa cells or granulosa cell precursors. Alternatively, or additionally, an increase in granulosa cell number in ovarian tissue is measured by comparison of the number of granulosa cells in the ovarian tissue after contact with granulosa cells or granulosa cell precursors as compared to age-matched ovarian tissue not contacted with granulosa cells or granulosa cell precursors. In some embodiments, the increase in granulosa cell number in ovarian tissue contacted with T regulatory cells and/or mesenchymal stem cells and/or granulosa cells or granulosa cell precursors is measured as a percent increase of about 1%, 5%, 10%, 20%, 30%, 40%, 50%, 60%, 70%, 80%, 90%, 100% or a percent increase between any two of these values as compared to, e.g., ovarian tissue before contact with granulosa cells or granulosa cell precursors or age-matched ovarian tissue not contacted with granulosa cells or granulosa cell precursors. Steroid hormones produced by the contacting of granulosa cells or granulosa cell precursors with ovarian tissue include, but are not limited to, estradiol, estriol, estrone, pregnenolone, and progesterone. In some embodiments, the increase in steroid hormones produced in ovarian tissue contacted with granulosa cells or granulosa cell precursors is measured as a percent increase of about 1%, 5%, 10%, 20%, 30%, 40%, 50%, 60%, 70%, 80%, 90%, 100% or a percent increase between any two of these values as compared to, e.g., ovarian tissue before contact with granulosa cells or granulosa cell precursors or age-matched ovarian tissue not contacted with granulosa cells or granulosa cell precursors. In some embodiments, an effective amount of an enriched population of granulosa cells or granulosa cell precursors is administered to a subject to increase steroidal hormones. In some embodiments, the granulosa cells or granulosa cell precursors secrete steroidal hormones. Alternatively, or additionally, in some embodiments, the granulosa cells or granulosa cell precursors are stimulated to secrete steroidal hormones by stimulating agents. Steroidal hormones secreted by the granulosa cells or granulosa cell precursors include, but are not limited to, estradiol, estriol, estrone, pregnenolone, and progesterone. Stimulating agents include, but are not limited to, follicle-stimulating hormone (FSH), 8-Bromoadenosine 3′,5′-cyclic monophosphate (8-br-cAMP), and luteinizing hormone (LH). In some embodiments, the granulosa cells or granulosa cell precursors are stimulated before administration to the subject, i.e., the granulosa cells or granulosa cell precursors are stimulated ex vivo to secrete steroidal hormones. In some embodiments, the granulosa cells or granulosa cell precursors are stimulated after administration to the subject, i.e., the granulosa cells or granulosa cell precursors are stimulated in vivo to secrete steroidal hormones. In some embodiments, the enriched population of granulosa cells or granulosa cell precursors is autologous to the subject. In some embodiments, the enriched population of granulosa cells or granulosa cell precursors is heterologous to the subject. In some embodiments, the subject suffers from reduced or lack of secretion of steroidal hormones. In some embodiments, the reduced or lack of secretion of steroidal hormones is due to menopause, ovariectomy, hysterectomy, premature ovarian failure, primary ovarian insufficiency, chemotherapy-induced ovarian failure, Turner's syndrome. In some embodiments, an increase in steroidal hormones in a subject in need thereof is based on a comparison between steroidal hormone levels in the subject before administration of the granulosa cells or granulosa cell precursors to steroidal hormone levels in the subject after administration of the granulosa cells or granulosa cell precursors. In some embodiments, an increase in steroidal hormones in a subject is based on the steroidal hormone levels in a subject after administration of granulosa cells or granulosa cell precursors as compared to steroidal hormone levels in a subject, who is sex and aged matched to the treated subject and not administered granulosa cells or granulosa cell precursors. In some embodiments, the increase in steroidal hormones produced in a subject administered granulosa cells or granulosa cell precursors is measured as a percent increase of about 1%, 5%, 10%, 20%, 30%, 40%, 50%, 60%, 70%, 80%, 90%, 100% or a percent increase between any two of these values as compared to, e.g., the subject before contacting with granulosa cells or granulosa cell precursors or a sex and aged matched subject not administered granulosa cells or granulosa cell precursors.

As with the T cells, T regulatory cells, regenerative cells, the effective amount of granulosa cells or granulosa cell precursors may be determined during pre-clinical trials and clinical trials by methods familiar to physicians and clinicians. An effective amount of granulosa cells or granulosa cell precursors useful in the methods may be administered to a subject in need thereof by any of a number of well-known methods for administering cells. The dose and/or dosage regimen will depend upon the characteristics of the condition being treated, e.g., the subject is in menopause or the subject had a hysterectomy, the subject, and the subject's history.

Any method known to those in the art for administration of cells as a therapy may be employed. In some embodiments, the T cells, T regulatory cells, regenerative cells, and/or stem cells and/or granulosa cells or granulosa cell precursors are administered to the subject, e.g., localized injection (e.g., catheter administration or direct intra-ovarian injection), systemic injection, intravenous injection, intrauterine injection, and parenteral administration. By way of example, but not by limitation, in some embodiments, granulosa cells or granulosa cell precursors are directly injected into ovarian tissue or ovaries.

### REFERENCES

ADDIN EN.REFLIST 1. Zhang, J., et al., The protective effects of human umbilical cord mesenchymal stem cells on damaged ovarian function: A comparative study. Biosci Trends, 2016. 10(4): p. 265-76.
2. Shen, J., D. Cao, and J. L. Sun, Ability of human umbilical cord mesenchymal stem cells to repair chemotherapy-induced premature ovarian failure. World J Stem Cells, 2020. 12(4): p. 277-287.
3. Zhao, Y., et al., Human umbilical cord mesenchymal stem cells restore the ovarian metabolome and rescue premature ovarian insufficiency in mice. Stem Cell Res Ther, 2020. 11(1): p. 466.
4. Liu, Q., et al., The effects of human umbilical cord mesenchymal stem cell transplantation on female fertility restoration in mice. Curr Gene Ther, 2021.
5. Wang, Z., et al., Mesenchymal Stem Cell Therapy Using Human Umbilical Cord in a Rat Model of Autoimmune-Induced Premature Ovarian Failure. Stem Cells Int, 2020. 2020: p. 3249495.
6. Zhang, X., et al., Human umbilical cord mesenchymal stem cells (hUCMSCs) promotes the recovery of ovarian function in a rat model of premature ovarian failure (POF). Gynecol Endocrinol, 2021. 37(4): p. 353-357.
7. Yang, Y., et al., Transplantation of umbilical cord-derived mesenchymal stem cells on a collagen scaffold improves ovarian function in a premature ovarian failure model of mice. In Vitro Cell Dev Biol Anim, 2019. 55(4): p. 302-311.
8. Zhou, Y., et al., Matrigel/Umbilical Cord-Derived Mesenchymal Stem Cells Promote Granulosa Cell Proliferation and Ovarian Vascularization in a Mouse Model of Premature Ovarian Failure. Stem Cells Dev, 2021. 30(15): p. 782-796.
9. Zheng, Q., et al., Umbilical Cord Mesenchymal Stem Cell Transplantation Prevents Chemotherapy-Induced Ovarian Failure via the NGF/TrkA Pathway in Rats. Biomed Res Int, 2019. 2019: p. 6539294.
10. Pan, X. H., et al., Effects and mechanisms of mUCMSCs on ovarian structure and function in naturally ageing C57 mice. J Ovarian Res, 2021. 14(1): p. 133.
11. Lu, X., et al., The effects of human umbilical cord-derived mesenchymal stem cell transplantation on endometrial receptivity are associated with Th1/Th2 balance change and uNK cell expression of uterine in autoimmune premature ovarian failure mice. Stem Cell Res Ther, 2019. 10(1): p. 214.
12. Deng, T., et al., Human Umbilical Cord Mesenchymal Stem Cells Improve Ovarian Function in Chemotherapy-Induced Premature Ovarian Failure Mice Through Inhibiting Apoptosis and Inflammation via a Paracrine Mechanism. Reprod Sci, 2021. 28(6): p. 1718-1732.
13. Lv, X., et al., Effects of single and multiple transplantations of human umbilical cord mesenchymal stem cells on the recovery of ovarian function in the treatment of premature ovarian failure in mice. J Ovarian Res, 2021. 14(1): p. 119.
14. Wang, J., et al., Activated Human Umbilical Cord Blood Platelet-Rich Plasma Enhances the Beneficial Effects of Human Umbilical Cord Mesenchymal Stem Cells in Chemotherapy-Induced POF Rats. Stem Cells Int, 2021. 2021: p. 8293699.
15. Zhao, T. X., et al., Low-dose interleukin-2 in patients with stable ischaemic heart disease and acute coronary syndromes (LILACS): protocol and study rationale for a randomised, double-blind, placebo-controlled, phase I/II clinical trial. BMJ Open, 2018. 8(9): p. e022452.
16. Jyonouchi, S., et al., Phase I trial of low-dose interleukin 2 therapy in patients with Wiskott-Aldrich syndrome. Clin Immunol, 2017. 179: p. 47-53.
17. Asano, T., et al., Phase I/IIa Study of Low Dose Subcutaneous Interleukin-2 (IL-2) for Treatment of Refractory Chronic Graft Versus Host Disease. Acta Med Okayama, 2016. 70(5): p. 429-433.
18. Kennedy-Nasser, A. A., et al., Ultra low-dose IL-2 for GVHD prophylaxis after allogeneic hematopoietic stem cell transplantation mediates expansion of regulatory T cells without diminishing antiviral and antileukemic activity. Clin Cancer Res, 2014. 20(8): p. 2215-25.
19. Mizui, M. and G. C. Tsokos, Low-Dose IL-2 in the Treatment of Lupus. Curr Rheumatol Rep, 2016. 18(11): p. 68.
20. Todd, J. A., et al., Regulatory T Cell Responses in Participants with Type 1 Diabetes after a Single Dose of Interleukin-2: A Non-Randomised, Open Label, Adaptive Dose-Finding Trial. PLoS Med, 2016. 13(10): p. e1002139.
21. Pham, M. N., M. G. von Herrath, and J. L. Vela, Antigen-Specific Regulatory T Cells and Low Dose of IL-2 in Treatment of Type 1 Diabetes. Front Immunol, 2015. 6: p. 651.
22. Waldron-Lynch, F., et al., Rationale and study design of the Adaptive study of IL-2 dose on regulatory T cells in type 1 diabetes (DILTID): a non-randomised, open label, adaptive dose finding trial. BMJ Open, 2014. 4(6): p. e005559.
23. von Bahr, V., et al., Mesenchymal stem cells may ameliorate inflammation in an ex vivo model of extracorporeal membrane oxygenation. Perfusion, 2019. 34(1_suppl): p. 15-21.
24. Huppert, L. A., K. D. Liu, and M. A. Matthay, Therapeutic potential of mesenchymal stromal cells in the treatment of ARDS. Transfusion, 2019. 59(51): p. 869-875.
25. Jung, Y. J., et al., The effect of human adipose-derived stem cells on lipopolysaccharide-induced acute respiratory distress syndrome in mice. Ann Transl Med, 2019. 7(22): p. 674.
26. Chen, C. H., et al., Effective protection against acute respiratory distress syndrome/sepsis injury by combined adipose-derived mesenchymal stem cells and preactivated disaggregated platelets. Oncotarget, 2017. 8(47): p. 82415-82429.
27. Lu, H., et al., Pulmonary Retention of Adipose Stromal Cells Following Intravenous Delivery Is Markedly Altered in the Presence of ARDS. Cell Transplant, 2016. 25(9): p. 1635-1643.
28. Zheng, G., et al., Treatment of acute respiratory distress syndrome with allogeneic adipose-derived mesenchymal stem cells: a randomized, placebo-controlled pilot study. Respir Res, 2014. 15: p. 39.
29. Lu, Z., et al., Mesenchymal stem cells induce dendritic cell immune tolerance via paracrine hepatocyte growth factor to alleviate acute ovarian injury. Stem Cell Res Ther, 2019. 10(1): p. 372.
30. Silva, J. D., et al., Mesenchymal Stromal Cells Are More Effective Than Their Extracellular Vesicles at Reducing Ovarian Injury Regardless of Acute Respiratory Distress Syndrome Etiology. Stem Cells Int, 2019. 2019: p. 8262849.
31. Xu, A. L., et al., Mesenchymal Stem Cells Reconditioned in Their Own Serum Exhibit Augmented Therapeutic Properties in the Setting of Acute Respiratory Distress Syndrome. Stem Cells Transl Med, 2019. 8(10): p. 1092-1106.
32. Cardenes, N., et al., Cell therapy for ARDS: efficacy of endobronchial versus intravenous administration and biodistribution of MAPCs in a large animal model. BMJ Open Respir Res, 2019. 6(1): p. e000308.
33. Li, L., et al., Mesenchymal stem cells with downregulated Hippo signaling attenuate ovarian injury in mice with lipopolysaccharideinduced acute respiratory distress syndrome. Int J Mol Med, 2019. 43(3): p. 1241-1252.
34. Mokhber Dezfouli, M. R., et al., Intrapulmonary autologous transplant of bone marrow-derived mesenchymal stromal cells improves lipopolysaccharide-induced acute respiratory distress syndrome in rabbit. Crit Care, 2018. 22(1): p. 353.
35. Schwede, M., et al., Effects of bone marrow-derived mesenchymal stromal cells on gene expression in human alveolar type II cells exposed to TNF-alpha, IL-1beta, and IFN-gamma. Physiol Rep, 2018. 6(16): p. e13831.
36. Masterson, C., et al., Syndecan-2-positive, Bone Marrow-derived Human Mesenchymal Stromal Cells Attenuate Bacterial-induced Acute Ovarian Injury and Enhance Resolution of Ventilator-induced Ovarian Injury in Rats. Anesthesiology, 2018. 129(3): p. 502-516.
37. Park, J., et al., Expression profile of microRNAs following bone marrow-derived mesenchymal stem cell treatment in lipopolysaccharide-induced acute ovarian injury. Exp Ther Med, 2018. 15(6): p. 5495-5502.
38. Pedrazza, L., et al., Mesenchymal stem cells improves survival in LPS-induced acute ovarian injury acting through inhibition of NETs formation. J Cell Physiol, 2017. 232(12): p. 3552-3564.
39. Yang, Y., et al., The Vascular Endothelial Growth Factors-Expressing Character of Mesenchymal Stem Cells Plays a Positive Role in Treatment of Acute Ovarian Injury In Vivo. Mediators Inflamm, 2016. 2016: p. 2347938.
40. Moodley, Y., et al., Human mesenchymal stem cells attenuate early damage in a ventilated pig model of acute ovarian injury. Stem Cell Res, 2016. 17(1): p. 25-31.
41. Hayes, M., et al., Mesenchymal stromal cells are more effective than the MSC secretome in diminishing injury and enhancing recovery following ventilator-induced ovarian injury. Intensive Care Med Exp, 2015. 3(1): p. 29.
42. Monsel, A., et al., Therapeutic Effects of Human Mesenchymal Stem Cell-derived Microvesicles in Severe Pneumonia in Mice. Am J Respir Crit Care Med, 2015. 192(3): p. 324-36.
43. Hao, Q., et al., Study of Bone Marrow and Embryonic Stem Cell-Derived Human Mesenchymal Stem Cells for Treatment of Escherichia coli Endotoxin-Induced Acute Ovarian Injury in Mice. Stem Cells Transl Med, 2015. 4(7): p. 832-40.
44. Devaney, J., et al., Human mesenchymal stromal cells decrease the severity of acute ovarian injury induced by E. coli in the rat. Thorax, 2015. 70(7): p. 625-35.
45. Asmussen, S., et al., Human mesenchymal stem cells reduce the severity of acute ovarian injury in a sheep model of bacterial pneumonia. Thorax, 2014. 69(9): p. 819-25.
46. Shalaby, S. M., et al., Mesenchymal stromal cell injection protects against oxidative stress in Escherichia coli-induced acute ovarian injury in mice. Cytotherapy, 2014. 16(6): p. 764-75.
47. Bustos, M. L., et al., Activation of human mesenchymal stem cells impacts their therapeutic abilities in ovarian injury by increasing interleukin (IL)-10 and IL-1RN levels. Stem Cells Transl Med, 2013. 2(11): p. 884-95.
48. Rojas, M., et al., Infusion of freshly isolated autologous bone marrow derived mononuclear cells prevents endotoxin-induced ovarian injury in an ex-vivo perfused swine model. Stem Cell Res Ther, 2013. 4(2): p. 26.
49. Yan, X., et al., Nrf2/Keap1/ARE Signaling Mediated an Antioxidative Protection of Human Placental Mesenchymal Stem Cells of Fetal Origin in Alveolar Epithelial Cells. Oxid Med Cell Longev, 2019. 2019: p. 2654910.
50. Cui, P., et al., Human amnion-derived mesenchymal stem cells alleviate ovarian injury induced by white smoke inhalation in rats. Stem Cell Res Ther, 2018. 9(1): p. 101.
51. Zhang, S., et al., Nrf2 transfection enhances the efficacy of human amniotic mesenchymal stem cells to repair ovarian injury induced by lipopolysaccharide. J Cell Biochem, 2018. 119(2): p. 1627-1636.
52. Huang, Z., et al., Transcriptomic analysis of ovarian tissues after hUC-MSCs and FTY720 treatment of lipopolysaccharide-induced acute ovarian injury in mouse models. Int Immunopharmacol, 2018. 63: p. 26-34.
53. Xuan, Y. Y., et al., Human Mesenchymal Stem/Stromal Cells From Human Umbilical Cord Ameliorate Acute Respiratory Distress Syndrome in Rats: Factors to Consider. Crit Care Med, 2017. 45(7): p. e736-e737.
54. Lee, F. Y., et al., Xenogeneic human umbilical cord-derived mesenchymal stem cells reduce mortality in rats with acute respiratory distress syndrome complicated by sepsis. Oncotarget, 2017. 8(28): p. 45626-45642.
55. Zhu, H., et al., Therapeutic Effects of Human Umbilical Cord-Derived Mesenchymal Stem Cells in Acute Ovarian Injury Mice. Sci Rep, 2017. 7: p. 39889.
56. Curley, G. F., et al., Cryopreserved, Xeno-Free Human Umbilical Cord Mesenchymal Stromal Cells Reduce Ovarian Injury Severity and Bacterial Burden in Rodent Escherichia coli-Induced Acute Respiratory Distress Syndrome. Crit Care Med, 2017. 45(2): p. e202-e212.
57. Chang, Y., et al., Intratracheal administration of umbilical cord blood-derived mesenchymal stem cells in a patient with acute respiratory distress syndrome. J Korean Med Sci, 2014. 29(3): p. 438-40.
58. Moodley, Y., et al., Human umbilical cord mesenchymal stem cells reduce fibrosis of bleomycin-induced ovarian injury. Am J Pathol, 2009. 175(1): p. 303-13.
59. Xiang, B., et al., Transplantation of Menstrual Blood-Derived Mesenchymal Stem Cells Promotes the Repair of LPS-Induced Acute Ovarian Injury. Int J Mol Sci, 2017. 18(4).
60. Wang, L., et al., Ovarian-Resident Mesenchymal Stem Cells Promote Repair of LPS-Induced Acute Ovarian Injury via Regulating the Balance of Regulatory T cells and Th1 7 cells. Inflammation, 2019. 42(1): p. 199-210.
61. Silva, J. D., et al., Mesenchymal Stem Cells From Bone Marrow, Adipose Tissue, and Ovarian Tissue Differentially Mitigate Ovarian and Distal Organ Damage in Experimental Acute Respiratory Distress Syndrome. Crit Care Med, 2018. 46(2): p. e132-e140.
62. Su, V. Y., et al., Mesenchymal Stem Cell-Conditioned Medium Induces Neutrophil Apoptosis Associated with Inhibition of the NF-kappaB Pathway in Endotoxin-Induced Acute Ovarian Injury. Int J Mol Sci, 2019. 20(9).
63. Mohammadipoor, A., et al., Therapeutic potential of products derived from mesenchymal stem/stromal cells in pulmonary disease. Respir Res, 2018. 19(1): p. 218.
64. Lee, J. H., J. Park, and J. W. Lee, Therapeutic use of mesenchymal stem cell-derived extracellular vesicles in acute ovarian injury. Transfusion, 2019. 59(S1): p. 876-883.
65. Abreu, S. C., D. J. Weiss, and P. R. Rocco, Extracellular vesicles derivedfrom mesenchymal stromal cells: a therapeutic option in respiratory diseases? Stem Cell Res Ther, 2016. 7(1): p. 53.
66. Monsel, A., et al., Mesenchymal stem cell derived secretome and extracellular vesicles for acute ovarian injury and other inflammatory ovarian diseases. Expert Opin Biol Ther, 2016. 16(7): p. 859-71.
67. Liu, F. B., Q. Lin, and Z. W. Liu, A study on the role of apoptotic human umbilical cord mesenchymal stem cells in bleomycin-induced acute ovarian injury in rat models. Eur Rev Med Pharmacol Sci, 2016. 20(5): p. 969-82.
68. Chen, J., et al., Mesenchymal Stem Cell Conditioned Medium Promotes Proliferation and Migration of Alveolar Epithelial Cells under Septic Conditions In Vitro via the JNK-P38 Signaling Pathway. Cell Physiol Biochem, 2015. 37(5): p. 1830-46.
69. Ionescu, L., et al., Stem cell conditioned medium improves acute ovarian injury in mice: in vivo evidencefor stem cell paracrine action. Am J Physiol Ovarian Cell Mol Physiol, 2012. 303(11): p. L967-77.
70. Court, A. C., et al., Mitochondrial transfer from MSCs to T cells induces Treg differentiation and restricts inflammatory response. EMBO Rep, 2020. 21(2): p. e48052.
71. Liu, Y., et al., Human umbilical cord mesenchymal stem cells confer potent immunosuppressive effects in Sjogren's syndrome by inducing regulatory T cells. Mod Rheumatol, 2020: p. 1-11.
72. Fakhimi, M., et al., Helios, CD73 and CD39 Induction in Regulatory T Cells Exposed to Adipose Derived Mesenchymal Stem Cells. Cell J, 2020. 22(2): p. 236-244.
73. Khosravi, M., et al., Induction of CD4(+)CD25(+)FOXP3(+) regulatory T cells by mesenchymal stem cells is associated with modulation of ubiquitination factors and TSDR demethylation. Stem Cell Res Ther, 2018. 9(1): p. 273.
74. Roux, C., et al., Immunosuppressive Mesenchymal Stromal Cells Derived from Human-Induced Pluripotent Stem Cells Induce Human Regulatory T Cells In Vitro and In Vivo. Front Immunol, 2017. 8: p. 1991.
75. Miyagawa, I., et al., Regulatory Mechanism of The Induction of Regulatory T Cells through Growth Factors Released by Human Mesenchymal Stem Cells. Crit Rev Immunol, 2018. 38(6): p. 471-478.
76. Khosravi, M., et al., Induction of CD4(+)CD25(+)Foxp3(+) regulatory T cells by mesenchymal stem cells is associated with RUNX complex factors. Immunol Res, 2018. 66(1): p. 207-218.
77. Khosravi, M., et al., Mesenchymal stem cells can induce regulatory T cells via modulating miR-126a but not miR-10a. Gene, 2017. 627: p. 327-336.
78. Lee, H. J., et al., ICOSL expression in human bone marrow-derived mesenchymal stem cells promotes induction of regulatory T cells. Sci Rep, 2017. 7: p. 44486.
79. Chen, C., et al., Mesenchymal stem cells upregulate Treg cells via sHLA-G in SLE patients. Int Immunopharmacol, 2017. 44: p. 234-241.
80. Lim, J. Y., et al., Enhanced immunoregulation of mesenchymal stem cells by IL-10-producing type 1 regulatory T cells in collagen-induced arthritis. Sci Rep, 2016. 6: p. 26851.
81. Lee, E. S., et al., Adoptive Transfer of Treg Cells Combined with Mesenchymal Stem Cells Facilitates Repopulation of Endogenous Treg Cells in a Murine Acute GVHD Model. PLoS One, 2015. 10(9): p. e0138846.
82. Cahill, E. F., et al., Jagged-1 is required for the expansion of CD4+CD25+FoxP3+ regulatory T cells and tolerogenic dendritic cells by murine mesenchymal stromal cells. Stem Cell Res Ther, 2015. 6: p. 19.
83. Wang, Z. X., et al., Mesenchymal stem cells alleviate atherosclerosis by elevating number and function of CD4(+)CD25 (+)FOXP3 (+) regulatory T-cells and inhibiting macrophage foam cell formation. Mol Cell Biochem, 2015. 400(1-2): p. 163-72.
84. Takahashi, T., et al., Multipotent mesenchymal stromal cells synergize with costimulation blockade in the inhibition of immune responses and the induction of Foxp3+ regulatory T cells. Stem Cells Transl Med, 2014. 3(12): p. 1484-94.
85. Frazier, T. P., et al., Human adipose-derived stromal/stem cells induce functional CD4+CD25+FoxP3+CD127-regulatory T cells under low oxygen culture conditions. Stem Cells Dev, 2014. 23(9): p. 968-77.
86. Li, J. G., et al., Human mesenchymal stem cells elevate CD4+CD25+CD127low/-regulatory T cells of asthmatic patients via heme oxygenase-1. Iran J Allergy Asthma Immunol, 2013. 12(3): p. 228-35.
87. Luz-Crawford, P., et al., Mesenchymal stem cells generate a CD4+CD25+Foxp3+ regulatory T cell population during the differentiation process of Th1 and Th17 cells. Stem Cell Res Ther, 2013. 4(3): p. 65.
88. Melief, S. M., et al., Multipotent stromal cells induce human regulatory T cells through a novel pathway involving skewing of monocytes toward anti-inflammatory macrophages. Stem Cells, 2013. 31(9): p. 1980-91.
89. Erkers, T., et al., Decidual stromal cells promote regulatory T cells and suppress alloreactivity in a cell contact-dependent manner. Stem Cells Dev, 2013. 22(19): p. 2596-605.
90. Engela, A. U., et al., Human adipose-tissue derived mesenchymal stem cells induce functional de-novo regulatory T cells with methylated FOXP3 gene DNA. Clin Exp Immunol, 2013. 173(2): p. 343-54.
91. Tasso, R., et al., Mesenchymal stem cells induce functionally active T-regulatory lymphocytes in a paracrine fashion and ameliorate experimental autoimmune uveitis. Invest Ophthalmol Vis Sci, 2012. 53(2): p. 786-93.
92. Choi, Y. S., J. A. Jeong, and D. S. Lim, Mesenchymal stem cell-mediated immature dendritic cells induce regulatory T cell-based immunosuppressive effect. Immunol Invest, 2012. 41(2): p. 214-29.
93. Sun, J., et al., Intrapulmonary delivery of human umbilical cord mesenchymal stem cells attenuates acute ovarian injury by expanding CD4+CD25+ Forkhead Boxp3 (FOXP3)+ regulatory T cells and balancing anti-and pro-inflammatory factors. Cell Physiol Biochem, 2011. 27(5): p. 587-96.
94. Madec, A. M., et al., Mesenchymal stem cells protect NOD mice from diabetes by inducing regulatory T cells. Diabetologia, 2009. 52(7): p. 1391-9.
95. English, K., et al., Cell contact, prostaglandin E(2) and transforming growth factor beta 1 play non-redundant roles in human mesenchymal stem cell induction of CD4⁺CD25(High) forkhead box P3+ regulatory T cells. Clin Exp Immunol, 2009. 156(1): p. 149-60.
96. Gonzalez-Rey, E., et al., Human adipose-derived mesenchymal stem cells reduce inflammatory and T cell responses and induce regulatory T cells in vitro in rheumatoid arthritis. Ann Rheum Dis, 2010. 69(1): p. 241-8.
97. Casiraghi, F., et al., Pretransplant infusion of mesenchymal stem cells prolongs the survival of a semiallogeneic heart transplant through the generation of regulatory T cells. J Immunol, 2008. 181(6): p. 3933-46.
98. Prevosto, C., et al., Generation of CD4+ or CD8+ regulatory T cells upon mesenchymal stem cell-lymphocyte interaction. Haematologica, 2007. 92(7): p. 881-8.
99. Hirsch, R., J. D. Ellenhorn, and J. A. Bluestone, In vivo administration of anti-CD3 monoclonal antibody can activate immune responses thus preventing malignant tumor growth. Princess Takamatsu Symp, 1988. 19: p. 237-43.
100. Schoof, D. D., et al., Activation of human tumor-infiltrating lymphocytes by monoclonal antibodies directed to the CD3 complex. Cancer Res, 1990. 50(4): p. 1138-43.
101. Davis, L. S., M. C. Wacholtz, and P. E. Lipsky, The induction of T cell unresponsiveness by rapidly modulating CD3. J Immunol, 1989. 142(4): p. 1084-94.
102. Anasetti, C., et al., Induction of specific nonresponsiveness in unprimed human T cells by anti-CD3 antibody and alloantigen. J Exp Med, 1990. 172(6): p. 1691-700.
103. Woodle, E. S., et al., Phase I trial of a humanized, Fc receptor nonbinding OKT3 antibody, huOKT3gammal(Ala-Ala) in the treatment of acute renal allograft rejection. Transplantation, 1999. 68(5): p. 608-16.
104. Cosimi, A. B., et al., Treatment of acute renal allograft rejection with OKT3 monoclonal antibody. Transplantation, 1981. 32(6): p. 535-9.
105. Ortho Multicenter Transplant Study, G., A randomized clinical trial of OKT3 monoclonal antibody for acute rejection of cadaveric renal transplants. N Engl J Med, 1985. 313(6): p. 337-42.
106. Debure, A., et al., One-month prophylactic use of OKT3 in cadaver kidney transplant recipients. Transplantation, 1988. 45(3): p. 546-53.
107. Oh, H. K., et al., Two low-dose OKT3 induction regimens following renal transplantation—clinical experience at a single center. Clin Transplant, 1998. 12(4): p. 343-7.
108. Opelz, G., Efficacy of rejection prophylaxis with OKT3 in renal transplantation. Collaborative Transplant Study. Transplantation, 1995. 60(11): p. 1220-4.
109. Darby, C. R., et al., Reduced dose OKT3 prophylaxis in sensitised kidney recipients. Transpl Int, 1996. 9(6): p. 565-9.
110. Ciancio, G., et al., Human donor bone marrow cells can enhance hyporeactivity in renal transplantation using maintenance FK 506 and OKT3 induction therapy. Transplant Proc, 1996. 28(2): p. 943-4.
111. Waid, T. H., et al., Treatment of renal allograft rejection with T10B9.1A31 or OKT3: final analysis of a phase II clinical trial. Transplantation, 1997. 64(2): p. 274-81.
112. Kumar, M. S., et al., ATGAM versus OKT3 induction therapy in cadaveric kidney transplantation: patient and graft survival, CD3 subset, infection, and cost analysis. Transplant Proc, 1998. 30(4): p. 1351-2.
113. Cosimi, A. B., et al., A randomized clinical trial comparing OKT3 and steroids for treatment of hepatic allograft rejection. Transplantation, 1987. 43(1): p. 91-5.
114. Millis, J. M., et al., Randomized prospective trial of OKT3 for early prophylaxis of rejection after liver transplantation. Transplantation, 1989. 47(1): p. 82-8.
115. Whiting, J. F., et al., Use of low-dose OKT3 as induction therapy in liver transplantation. Transplantation, 1998. 65(4): p. 577-80.
116. Melzer, J. S., et al., The use of OKT3 in combined pancreas-kidney allotransplantation. Transplant Proc, 1990. 22(2): p. 634-5.
117. Sindhi, R., et al., Increased risk of pulmonary edema in diabetic patients undergoing preemptive pancreas transplantation with OKT3 induction. Transplant Proc, 1995. 27(6): p. 3016-7.
118. Stratta, R. J., et al., A prospective randomized trial of OKT3 vs ATGAM induction therapy in pancreas transplant recipients. Transplant Proc, 1996. 28(2): p. 917-8.
119. Ross, D. J., et al., Delayed development of obliterative bronchiolitis syndrome with OKT3 after unilateral ovarian transplantation. A plea for multicenter immunosuppressive trials. Chest, 1996. 109(4): p. 870-3.
120. van Gelder, T., et al., A randomized trial comparing safety and efficacy of OKT3 and a monoclonal anti-interleukin-2 receptor antibody (BT563) in the prevention of acute rejection after heart transplantation. Transplantation, 1996. 62(1): p. 51-5.
121. Delgado, J. F., et al., Induction treatment with monoclonal antibodies for heart transplantation. Transplant Rev (Orlando), 2011. 25(1): p. 21-6.
122. Kormos, R. L., et al., Monoclonal versus polyclonal antibody therapy for prophylaxis against rejection after heart transplantation. J Heart Transplant, 1990. 9(1): p. 1-9, discussion 9-10.
123. Rabinov, M., et al., Recipient selection algorithm for immunosuppression in cardiac transplantation: OKT3 vs triple therapy alone. Transplant Proc, 1992. 24(1): p. 167-8.
124. Chin, C., et al., Induction therapy for pediatric and adult heart transplantation: comparison between OKT3 and daclizumab. Transplantation, 2005. 80(4): p. 477-81.
125. Prentice, H. G., et al., Use of anti-T-cell monoclonal antibody OKT3 to prevent acute graft-versus-host disease in allogeneic bone-marrow transplantation for acute leukaemia. Lancet, 1982. 1(8274): p. 700-3.
126. Weinshenker, B. G., et al., An open trial of OKT3 in patients with multiple sclerosis. Neurology, 1991. 41(7): p. 1047-52.
127. Herold, K. C., et al., A single course of anti-CD3 monoclonal antibody hOKT3gammal(Ala-Ala) results in improvement in C-peptide responses and clinical parameters for at least 2 years after onset of type 1 diabetes. Diabetes, 2005. 54(6): p. 1763-9.
128. Ferran, C., et al., Cytokine-related syndrome following injection of anti-CD3 monoclonal antibody: further evidence for transient in vivo T cell activation. Eur J Immunol, 1990. 20(3): p. 509-15.
129. Vasquez, E. M., A. J. Fabrega, and R. Pollak, OKT3-induced cytokine-release syndrome: occurrence beyond the second dose and association with rejection severity. Transplant Proc, 1995. 27(1): p. 873-4.
130. Norman, D. J., J. A. Kimball, and J. M. Barry, Cytokine-release syndrome: differences between high and low doses of OKT3. Transplant Proc, 1993. 25(2 Suppl 1): p. 35-8.
131. Goldman, M., et al., OKT3-induced cytokine release attenuation by high-dose methylprednisolone. Lancet, 1989. 2(8666): p. 802-3.
132. Fletcher, E. A. K., et al., Extracorporeal human whole blood in motion, as a tool to predict first-infusion reactions and mechanism-of-action of immunotherapeutics. Int Immunopharmacol, 2018.54: p. 1-11.
133. Chatenoud, L., et al., In vivo cell activation following OKT3 administration. Systemic cytokine release and modulation by corticosteroids. Transplantation, 1990. 49(4): p. 697-702.
134. Chatenoud, L., et al., Corticosteroid inhibition of the OKT3-induced cytokine-related syndrome—dosage and kinetics prerequisites. Transplantation, 1991. 51(2): p. 334-8.
135. Bugelski, P. J., et al., Monoclonal antibody-induced cytokine-release syndrome. Expert Rev Clin Immunol, 2009. 5(5): p. 499-521.
136. Barel, D., et al., Enhanced tumor necrosis factor in anti-CD3 antibody stimulated diabetic NOD mice: modulation by PGE1 and dietary lipid. Autoimmunity, 1992. 13(2): p. 141-9.
137. Pradier, O., et al., Procoagulant effect of the OKT3 monoclonal antibody: involvement of tumor necrosis factor. Kidney Int, 1992. 42(5): p. 1124-9.
138. Wissing, K. M., et al., A pilot trial of recombinant human interleukin-10 in kidney transplant recipients receiving OKT3 induction therapy. Transplantation, 1997. 64(7): p. 999-1006.
139. Charpentier, B., et al., Evidence that antihuman tumor necrosis factor monoclonal antibody prevents OKT3-induced acute syndrome. Transplantation, 1992. 54(6): p. 997-1002.
140. DeVault, G. A., Jr., et al., The effects of oral pentoxifylline on the cytokine release syndrome during inductive OKT3. Transplantation, 1994. 57(4): p. 532-40.
141. Ilan, Y., et al., Oral administration of OKT3 monoclonal antibody to human subjects induces a dose-dependent immunologic effect in T cells and dendritic cells. J Clin Immunol, 2010. 30(1): p. 167-77.
142. Lalazar, G., et al., Oral Administration of OKT3 MAb to Patients with NASH, Promotes Regulatory T-cell Induction, and Alleviates Insulin Resistance: Results of a Phase IIa Blinded Placebo-Controlled Trial. J Clin Immunol, 2015. 35(4): p. 399-407.
143. Roten, R., et al., Plasma levels of tumor necrosis factor in the adult respiratory distress syndrome. Am Rev Respir Dis, 1991. 143(3): p. 590-2.
144. Sheng, W. H., et al., Clinical manifestations and inflammatory cytokine responses in patients with severe acute respiratory syndrome. J Formos Med Assoc, 2005. 104(10): p. 715-23.
145. Romaschin, A. D., et al., Systemic phospholipase A2 and cachectin levels in adult respiratory distress syndrome and multiple-organ failure. Clin Biochem, 1992. 25(1): p. 55-60.
146. Koh, Y., et al., Tumor necrosis factor induced acute ovarian leak in rats: less than with interleukin-1. Inflammation, 1996. 20(5): p. 461-9.
147. Dunican, A. L., et al., TNFalpha-induced suppression of PMN apoptosis is mediated through interleukin-8 production. Shock, 2000. 14(3): p. 284-8; discussion 288-9.
148. Negrier, M. S., et al., Phase I trial of recombinant interleukin-2 followed by recombinant tumor necrosis factor in patients with metastatic cancer. J Immunother (1991), 1992. 11(2): p. 93-102.
149. Malemud, C. J., Recent advances in neutralizing the IL-6 pathway in arthritis. Open Access Rheumatol, 2009. 1: p. 133-150.
150. Romagnani, S., Human Th17 cells. Arthritis Res Ther, 2008. 10(2): p. 206.
151. Romagnani, S., et al., Properties and origin of human Th17 cells. Mol Immunol, 2009. 47(1): p. 3-7.
152. Kimura, A. and T. Kishimoto, IL-6: regulator of Treg/Th17 balance. Eur J Immunol, 2010. 40(7): p. 1830-5.
153. Yu, Z. X., et al., The ratio of Th17/Treg cells as a risk indicator in early acute respiratory distress syndrome. Crit Care, 2015. 19: p. 82.
154. Mock, J. R., et al., Foxp3+ regulatory T cells promote ovarian epithelial proliferation. Mucosal Immunol, 2014. 7(6): p. 1440-51.
155. Kimura, A. and T. Kishimoto, Th17 cells in inflammation. Int Immunopharmacol, 2011. 11(3): p. 319-22.
156. Meduri, G. U., et al., Persistent elevation of inflammatory cytokines predicts a poor outcome in ARDS. Plasma IL-1 beta and IL-6 levels are consistent and efficient predictors of outcome over time. Chest, 1995. 107(4): p. 1062-73.
157. Hui, L., et al., Higher serum procalcitonin and IL-6 levels predict worse diagnosis for acute respiratory distress syndrome patients with multiple organ dysfunction. Int J Clin Exp Pathol, 2017. 10(7): p. 7401-7407.
158. Swaroopa, D., et al., Association of serum interleukin-6, interleukin-8, and Acute Physiology and Chronic Health Evaluation II score with clinical outcome in patients with acute respiratory distress syndrome. Indian J Crit Care Med, 2016. 20(9): p. 518-25.
159. Bime, C., et al., Development of a biomarker mortality risk model in acute respiratory distress syndrome. Crit Care, 2019. 23(1): p. 410.
160. Spadaro, S., et al., Biomarkers for Acute Respiratory Distress syndrome and prospects for personalised medicine. J Inflamm (Lond), 2019. 16: p. 1.
161. Nakamoto, S., Reflections on My Lifetime Teacher. Dr. Willem J. Kolff. Artif Organs, 2018. 42(2): p. 115-126.
162. Krakauer, R. S., et al., Circulating immune complexes in rheumatoid arthritis. Selective removal by cryogelation with membrane filtration. Arch Intern Med, 1982. 142(2): p. 395-7.
163. Snyder, H. W., Jr., et al., Clearance of feline leukemia virus from persistently infected pet cats treated by extracorporeal immunoadsorption is correlated with an enhanced antibody response to FeLV gp 70. J Immunol, 1984. 132(3): p. 1538-43.
164. Hellstrom, K. E., et al., Blocking (suppressor)factors, immune complexes, and extracorporeal immunoadsorption in tumor immunity. Contemp Top Immunobiol, 1985. 15: p. 213-38.
165. Messerschmidt, G. L., et al., Protein A immunoadsorption in the treatment of malignant disease. J Clin Oncol, 1988. 6(2): p. 203-12.
166. Cameron, D. J., C. T. Fitts, and P. R. Rajagopalan, Antigen-coated immunoadsorbents utilized for in vivo depletion of antibodies and lymphocytes with specificity for the antigen. J Surg Oncol, 1983. 23(3): p. 158-62.
167. Lazaridis, K., et al., Specific removal of autoantibodies by extracorporeal immunoadsorption ameliorates experimental autoimmune myasthenia gravis. J Neuroimmunol, 2017. 312: p. 24-30.
168. Lupinek, C., et al., Extracorporeal IgE Immunoadsorption in Allergic Asthma: Safety and Efficacy. EBioMedicine, 2017. 17: p. 119-133.
169. Fabbrini, P., et al., Light chains removal by extracorporeal techniques in acute kidney injury due to multiple myeloma: a position statement of the Onconephrology Work Group of the Italian Society of Nephrology. J Nephrol, 2016. 29(6): p. 735-746.
170. Opgenoorth, M., et al., Treatment of antibody-mediated rejection including immunoadsorption during first year after renal transplantation—Clinical results and regulation of endothelial progenitor cells. Atheroscler Suppl, 2015. 18: p. 67-73.
171. Lazaridis, K., et al., Antigen-specific apheresis of autoantibodies in myasthenia gravis. Ann N Y Acad Sci, 2012. 1275: p. 7-12.
172. Koch, B., S. Buttner, and H. Geiger, [Reducing viral load in life-threatening viral diseases using snowdrops]. Dtsch Med Wochenschr, 2016. 141(25): p. 1868-1871.
173. Koch, B., et al., Lectin Affinity Plasmapheresis for Middle East Respiratory Syndrome-Coronavirus and Marburg Virus Glycoprotein Elimination. Blood Purif, 2018. 46(2): p. 126-133.
174. Moriyama, M., et al., Removal of hepatitis C virus by G-1 beads in sera from patients with chronic hepatitis C. Intervirology, 2005. 48(2-3): p. 84-8.
175. Josephs, S. F., et al., Unleashing endogenous TNF-alpha as a cancer immunotherapeutic. J Transl Med, 2018. 16(1): p. 242.
176. Kanekura, T., et al., Granulocyte and monocyte adsorption apheresis (GCAP)for refractory skin diseases caused by activated neutrophils and psoriatic arthritis: evidence that GCAP removes Mac-i-expressing neutrophils. Ther Apher Dial, 2006. 10(3): p. 247-56.
177. Nakane, S., et al., Cytapheresis with a filter for selective removal of CD4+ T cells in experimental autoimmune encephalomyelitis. Mult Scler, 2003. 9(6): p. 579-84.

1. A method of inhibiting ovarian failure and/or stimulating ovarian regeneration in a patient in need comprising administration of a T cell population expressing FoxP3 to said subject.

2. The method of claim 1, wherein said T cell population expresses TGF-beta protein in a membrane bound form.

3. The method of claim 1, wherein said T cell population expresses GITR and/or GITR ligand.

4. The method of claim 1, wherein said T cell population possesses ability to suppress a mixed lymphocyte reaction.

5. The method of claim 4, wherein said suppression of said mixed lymphocyte reaction is inhibition of cytokine production.

6. The method of claim 4, wherein said suppression of said mixed lymphocyte reaction is inhibition of interleukin-12 production.

7. The method of claim 4, wherein said suppression of said mixed lymphocyte reaction is inhibition of interleukin-15 production.

8. The method of claim 1, wherein said Foxp3 expressing cells express interleukin-3 receptor.

9. The method of claim 1, wherein said Foxp3 expressing cells express TGF-beta.

10. The method of claim 1, wherein said Foxp3 expressing cells express Helios.

11. The method of claim 1, wherein said Foxp3 expressing cells express CD25.

12. The method of claim 1, wherein said Foxp3 expressing cells express c-kit.

13. The method of claim 1, wherein said Foxp3 expressing cells express FGF-2.

14. The method of claim 1, wherein said Foxp3 expressing cells express OCT-2.

15. The method of claim 1, wherein said Foxp3 expressing cells express NANOG.

16. The method of claim 1, wherein said FoxP3 expressing cells are pretreated with an activator of indolamine 2-3 deoxygenase.

17. The method of claim 1, wherein ovarian failure is caused by chemotherapy or radiation therapy.

18. The method of claim 1, wherein said ovarian failure is caused by age.

19. The method of claim 1, wherein said ovarian failure is ovarian fibrosis.

20. The method of claim 1, where said ovarian failure is improved by mesenchymal cells.
